(19) United States
(12) Patent Application Publication
(10) Pub. No.: US 2024/0417692 A1
(43) Pub. Date: Dec. 19, 2024

(54) DIFFERENTIATION OF HUMAN EMBRYONIC STEM CELLS INTO PANCREATIC ENDOCRINE CELLS USING HB9 REGULATORS

(71) Applicant: Janssen Biotech, Inc., Horsham, PA (US)

(72) Inventor: Alireza Rezania, Wellesley, MA (US)

(73) Assignee: Janssen Biotech, Inc., Horsham, PA (US)

(21) Appl. No.: 18/627,889

(22) Filed: Apr. 5, 2024

Related U.S. Application Data
(63) Continuation of application No. 17/160,087, filed on Jan. 27, 2021, now abandoned.
Application No. 17/160,087 is a continuation of application No. 16/200,469, filed on Nov. 26, 2018, now Pat. No. 10,947,511.
Application No. 16/200,469 is a division of application No. 13/998,883, filed on Dec. 18, 2013, now Pat. No. 10,138,465.
(60) Provisional application No. 61/747,672, filed on Dec. 31, 2012.

(51) Int. Cl. C12N 5/071; C12N 5/0735

(52) U.S. Cl. CPC C12N 5/0676; C12N 5/0606; C12N 2501/117; C12N 2501/15; C12N 2501/16; C12N 2501/19; C12N 2501/395; C12N 2501/415; C12N 2501/727; C12N 2501/999; C12N 2506/02; C12N 2533/90

(57) ABSTRACT

The present invention provides methods to promote differentiation of pluripotent stem cells to pancreatic endoderm cells expressing PDX1, NKX6.1, and HB9. In particular, the methods encompass culturing Stage 4 to Stage 6 cells with a thyroid hormone (e.g. T3), an ALK5 inhibitor, or both.

### CROSS-REFERENCE TO RELATED APPLICATIONS

This is a continuation of U.S. patent application Ser. No. 17/160,087, filed on Jan. 27, 2021, which is a continuation of U.S. patent application Ser. No. 16/200,469, filed on Nov. 26, 2018, issued as U.S. Pat. No. 10,947,511, which is a divisional application of U.S. application Ser. No. 13/998,883, filed on Dec. 18, 2013, issued as U.S. Pat. No. 10,138,465, which claims priority to U.S. Provisional Application 61/747,672, filed on Dec. 31, 2012. The prior applications are incorporated by reference in their entireties.

### FIELD OF THE INVENTION

The present invention is in the field of cell differentiation. More specifically, the invention involves the use of specific thyroid hormones, or analogues thereof, and ALK5 inhibitors as regulators of HB9 in pancreatic endoderm and endocrine cells.

### BACKGROUND

Advances in cell-replacement therapy for Type I diabetes mellitus and a shortage of transplantable islets of Langerhans have focused interest on developing sources of insulin-producing cells, or β cells, appropriate for engraftment. One approach is the generation of functional β cells from pluripotent stem cells, such as, embryonic stem cells.

In vertebrate embryonic development, a pluripotent cell gives rise to a group of cells comprising three germ layers (ectoderm, mesoderm, and endoderm) in a process known as gastrulation. Tissues such as, thyroid, thymus, pancreas, gut, and liver, will develop from the endoderm, via an intermediate stage. The intermediate stage in this process is the formation of definitive endoderm.

By the end of gastrulation, the endoderm is partitioned into anterior-posterior domains that can be recognized by the expression of a panel of factors that uniquely mark anterior, mid, and posterior regions of the endoderm. For example, HHEX, and SOX2 identify the anterior region while CDX1, 2, and 4 identify the posterior region of the endoderm.

Migration of endoderm tissue brings the endoderm into close proximity with different mesodermal tissues that help in regionalization of the gut tube. This is accomplished by a plethora of secreted factors, such as FGFs, WNTs, TGF-βs, retinoic acid (RA), and BMP ligands and their antagonists. For example, FGF4 and BMP promote CDX2 expression in the presumptive hindgut endoderm and repress expression of the anterior genes HHEX and SOX2 (2000 Development, 127:1563-1567). WNT signaling has also been shown to work in parallel to FGF signaling to promote hindgut development and inhibit foregut fate (2007 Development, 134:2207-2217). Lastly, secreted retinoic acid by mesenchyme regulates the foregut-hindgut boundary (2002 Curr Biol, 12:1215-1220).

The level of expression of specific transcription factors may be used to designate the identity of a tissue. During transformation of the definitive endoderm into a primitive gut tube, the gut tube becomes regionalized into broad domains that can be observed at the molecular level by restricted gene expression patterns. The regionalized pancreas domain in the gut tube shows a very high expression of PDX1 and very low expression of CDX2 and SOX2. PDX1, NKX6.1, PTFIA, and NKX2.2 are highly expressed in pancreatic tissue; and expression of CDX2 is high in intestinal tissue.

Formation of the pancreas arises from the differentiation of definitive endoderm into pancreatic endoderm. Dorsal and ventral pancreatic domains arise from the foregut epithelium. Foregut also gives rise to the esophagus, trachea, lungs, thyroid, stomach, liver, and bile duct system.

Cells of the pancreatic endoderm express the pancreatic-duodenal homeobox gene PDX1. In the absence of PDX1, the pancreas fails to develop beyond the formation of ventral and dorsal buds. Thus, PDX1 expression marks a critical step in pancreatic organogenesis. The mature pancreas contains both exocrine and endocrine tissues arising from the differentiation of pancreatic endoderm.

D'Amour et al. describe the production of enriched cultures of human embryonic stem cell-derived definitive endoderm in the presence of a high concentration of activin and low serum (Nature Biotechnology 2005, 23:1534-1541; U.S. Pat. No. 7,704,738). Transplanting these cells under the kidney capsule of mice reportedly resulted in differentiation into more mature cells with characteristics of endodermal tissue (U.S. Pat. No. 7,704,738). Human embryonic stem cell-derived definitive endoderm cells can be further differentiated into PDX1 positive cells after addition of FGF10 and retinoic acid (U.S. Patent App. Pub. No. 2005/0266554). Subsequent transplantation of these pancreatic precursor cells in the fat pad of immune deficient mice resulted in the formation of functional pancreatic endocrine cells following a 3-4 months maturation phase (U.S. Pat. Nos. 7,993,920 and 7,534,608).

Fisk et al. report a system for producing pancreatic islet cells from human embryonic stem cells (U.S. Pat. No. 7,033,831). In this case, the differentiation pathway was divided into three stages. Human embryonic stem cells were first differentiated to endoderm using a combination of sodium butyrate and activin A (U.S. Pat. No. 7,326,572). The cells were then cultured with BMP antagonists, such as Noggin, in combination with EGF or betacellulin to generate PDX1 positive cells. The terminal differentiation was induced by nicotinamide.

Small molecule inhibitors have also been used for induction of pancreatic endocrine precursor cells. For example, small molecule inhibitors of TGF-β receptor and BMP receptors (Development 2011, 138:861-871; Diabetes 2011, 60:239-247) have been used to significantly enhance the number of pancreatic endocrine cells. In addition, small molecule activators have also been used to generate definitive endoderm cells or pancreatic precursor cells (Curr Opin Cell Biol 2009, 21:727-732; Nature Chem Biol 2009, 5:258-265).

HB9 (also known as HIXB9 and MNX1) is a bHLH transcriptional activator protein expressed early in pancreas development starting at approximately embryonic day 8. HB9 is also expressed in notochord and spinal cord. Expression of HB9 is transient and peaks at about day 10.5 in pancreatic epithelium being expressed in PDX1 and NKX6.1 expressing cells. At about day 12.5, HB9 expression declines and at later stages it becomes restricted only to β cells. In mice homozygous for a null mutation of HIXB9, the dorsal lobe of the pancreas fails to develop (Nat Genet 23:67-70, 1999; Nat Genet 23:71-75, 1999). HB9−/− β-cells express low levels of the glucose transporter, GLUT2, and NKX6.1. Furthermore, HB9−/− pancreas shows a significant reduction in the number of insulin positive cells while not significantly affecting expression of other pancreatic hormones. Thus, temporal control of HB9 is essential to normal β cell development and function. While not much is known about factors regulating HB9expression in β cells, a recent study in zebrafish suggests that retinoic acid can positively regulate expression of HB9 (Development, 138, 4597-4608, 2011).

The thyroid hormones, thyroxine (“T4”) and triiodothyronine (“T3”), are tyrosine-based hormones produced by the thyroid gland and are primarily responsible for regulation of metabolism. The major form of thyroid hormone in the blood is T4, which has a longer half-life than T3. The ratio of T4 to T3 released into the blood is roughly 20 to 1. T4 is converted to the more active T3 (three to four times more potent than T4) within cells by deiodinase.

T3 binds to thyroid hormone receptors, TRα1 nd TRβ1 (TR). TR is a nuclear hormone receptor, which heterodimerizes with retinoid X receptor. The dimers bind to the thyroid response elements (TREs) in the absence of ligand and act as transcriptional repressors. Binding of T3 to TR reduces the repression of TRE dependent genes and induces the expression of various target genes. While numerous studies have suggested a role for T3 in increasing β cell proliferation, reducing apoptosis, and improving insulin secretion, its role in cell differentiation is undefined.

Transforming growth factor β (TGF-β) is a member of a large family of pleiotropic cytokines that are involved in many biological processes, including growth control, differentiation, migration, cell survival, fibrosis and specification of developmental fate. TGF-β superfamily members signal through a receptor complex comprising a type II and type I receptor. TGF-B ligands (such as activins, and GDFs (growth differentiation factors)) bring together a type II receptor with a type I receptor. The type II receptor phosphorylates and activates the type I receptor in the complex. There are five mammalian type II receptors: TβR-II, ActR-II, ActR-IIB, BMPR-II, and AMHR-II and seven type I receptors (ALKs 1-7). Activin and related ligands signal via combinations of ActR-II or ActR-IIB and ALK4 or ALK5, and BMPs signal through combinations of ALK2, ALK3, and ALK6 with ActR-II, ActR-IIB, or BMPR-II. AMH signals through a complex of AMHR-II with ALK6, and nodal has been shown recently to signal through a complex of ActR-IIB and ALK7 (Cell. 2003,113(6):685-700). Following binding of the TGF-B ligand to the appropriate receptor, the ensuing signals are transduced to the nucleus primarily through activation of complexes of Smads. Upon activation, the type I receptors phosphorylate members of the receptor-regulated subfamily of Smads. This activates them and enables them to form complexes with a common mediator Smad, Smad4. Smads 1, 5, and 8 are substrates for ALKs 1, 2, 3, and 6, whereas Smads 2 and 3 are substrates for ALKs 4, 5, and 7 (FASEB J 13:2105-2124). The activated Smad complexes accumulate in the nucleus, where they are directly involved in the transcription of target genes, usually in association with other specific DNA-binding transcription factors. Compounds that selectively inhibit the receptors for TGF-β, have been developed for therapeutic applications and for modulating cell fate in the context of reprogramming and differentiation from various stem cell populations. In particular, ALK5 inhibitors have been previously used to direct differentiation of embryonic stem cells to an endocrine fate (Diabetes, 2011, 60(1):239-47).

In general, the process of differentiating progenitor cells to functional β cells goes through various stages. Yet it is recognized that directing human embryonic stem (“hES”) cells in vitro progressively through stages of commitment to cells resembling β-cells is challenging and production of functional β-cells from hES cells is not a straightforward process. Each step in the process of differentiating progenitor cells presents a unique challenge. Although progress has been made in improving protocols to generate pancreatic cells from progenitor cells such as human pluripotent stem cells, there is still a need to generate a protocol that results in functional endocrine cells and, in particular, β cells.

### BRIEF DESCRIPTION OF THE DRAWINGS

FIGS. 1A to 1C depict data from real-time PCR analyses of the expression of the following genes in cells of the human embryonic stem cell line H1 differentiated to pancreatic endoderm/endocrine precursors as outlined in Example 1: PDX1 (FIG. 1A); NKX6.1 (FIG. 1B); and HB9 (FIG. 1C).

FIGS. 2A to 2C show the results of FACS analysis of the human embryonic stem cell line H1 differentiated to pancreatic endoderm/endocrine precursors as outlined in Example 1 for PDX1 (FIG. 2A), NKX6.1 (FIG. 2B) and HB9 (FIG. 2C).

FIGS. 3A and 3B show images of cells immunostained for NXK6.1, insulin or HB9. The cells were differentiated to pancreatic endoderm/endocrine precursors as outlined in Example 1. FIG. 3A shows immune staining for NKX6.1 (left hand pane) and insulin (right hand pane). FIG. 3B shows immune staining for HB9 (left hand pane) and insulin (right hand pane).

FIGS. 4A, 4B, and 4C depict the FACS data for percent expression of PDX1, NKX6.1, and HB9 at Stage 4 day 3 (FIG. 4A), Stage 5 day 4 (FIG. 4B) and Stage 6 day 3 (FIG. 4C) of embryonic stem cell line H1 differentiated to Stages 4 through 6 as outlined in Example 2.

FIGS. 5A-5B are a bar graph and two digital images. FIG. 5A shows mRNA expression of HB9 as compared to human islets at Stages 2 through 6 for cells differentiated as outlined in Example 2.

FIG. 5B depicts images of Stage 4 day 3 cells, which were differentiated as outlined in Example 2, immunostained for NXK6.1 (left hand pane) and HB9 (right hand pane).

FIGS. 6A to 6J depict data from real-time PCR analyses of the expression of the following genes in cells of the human embryonic stem cell line H1 differentiated to Stage 4 as outlined in Example 2 and then treated at Stage 4 only, Stage 4 through Stage 5, or Stage 4 through Stage 6. FIGS. 6A to 6J depict the data for the following: NKX6.1 (FIG. 6A); PDX1 (FIG. 6B); NKX2.2 (FIG. 6C); glucagon (FIG. 6D); insulin (FIG. 6E); somatostatin (FIG. 6F); CDX2 (FIG. 6G); albumin (FIG. 6H); gastrin (FIG. 6I); and SOX2 (FIG. 6J).

FIGS. 7A and 7B show the results of immunostaining of control (FIG. 7A) and cultures treated (FIG. 7B) as outlined in Example 2. Immunostaining of control (FIG. 7A) and treated cultures (FIG. 7B) at Stage 6 revealed a significant increase in the number of HB9 positive cells in the T3 treated group (FIG. 7B) as compared to the control (FIG. 7A) at Stage 6.

FIGS. 8A and 8B depict immunostaining for NKX6.1 and HB9 at Stage 6 day 7 for cells differentiated to Stage 6 as outlined in Example 3. FIG. 8C depicts data from real-time PCR analyses of the expression of the HB9 in cells of the human embryonic stem cell line H1 differentiated to Stage 6 as outlined in Example 3.

FIGS. 9A and 9B depict the FACS data at Stage 6 day 5 and day 15, respectively, of the HB9 in cells of the human embryonic stem cell line H1 differentiated to Stage 6 as outlined in Example 3.

FIGS. 10A to 10E depict immunostaining for NKX6.1 and HB9 at Stage 6 day 6 for cells that were differentiated according to the protocol outlined in Example 4. T3 in a dose dependent manner significantly enhanced the number of HB9 positive cells in the NKX6.1 positive pancreatic endoderm precursor cells.

FIGS. 11A to 11L depict data from real-time PCR analyses of the expression of the following genes in cells of the human embryonic stem cell line H1 differentiated to Stage 6 as outlined in Example 4: SOX2 (FIG. 11A); NKX6.1 (FIG. 11B); NKX2.2 (FIG. 11C); gastrin (FIG. 11D); PDX1 (FIG. 11E); NGN3 (FIG. 11F); PAX6 (FIG. 11G); PAX4 (FIG. 11H); insulin (FIG. 11I); glucagon (FIG. 11J); ghrelin (FIG. 11K); and somatostatin (FIG. 11L).

### DETAILED DESCRIPTION

The following detailed description of the invention will be better understood when read in conjunction with the appended figures. Figures are provided for the purpose of illustrating certain embodiments of the invention. However, the invention is not limited to the precise arrangements, examples, and instrumentalities shown. For clarity of disclosure, and not by way of limitation, the detailed description of the invention is divided into subsections that describe or illustrate certain features, embodiments, or applications of the present invention.

The present invention pertains to the generation of pancreatic endoderm cells that are positive for NKX6.1, PDX1, and HB9 via use of certain thyroid hormones, or analogues thereof, and ALK5 (TGFB type I receptor kinase) inhibitors in a specific culturing sequence. Accordingly, the present invention provides an in vitro cell culture for differentiating cells derived from pluripotent stem cells into cells expressing markers characteristic of the β cell lineage that express NKX6.1, PDX1 and HB9. The invention further provides a method for obtaining such cells via an in vitro cell culture. In certain embodiments, the invention is based on the discovery that the inclusion of T3, T4, or analogues thereof, act as an inducer of HB9 protein expression in differentiating cells to facilitate differentiation towards β cells. HB9 is not expressed at the protein level at Stage 3 or Stage 4. Accordingly, the present invention provides methods of differentiating stem cells by regulating HB9 protein expression. In particular, this invention provides for the generation of pancreatic endoderm cells that are positive for NKX6.1, PDX1, and HB9 via use of T3 or T4, or analogues thereof, and ALK5 inhibition in a specific culturing sequence.

### Definitions

Stem cells are undifferentiated cells defined by their ability, at the single cell level, to both self-renew and differentiate. Stem cells may produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm, and ectoderm). Stem cells also give rise to tissues of multiple germ layers following transplantation and contribute substantially to most, if not all, tissues following injection into blastocysts.

Stem cells are classified by their developmental potential. Pluripotent stem cells are able to give rise to all embryonic cell types.

Differentiation is the process by which an unspecialized (“uncommitted”) or less specialized cell acquires the features of a specialized cell such as, for example, a nerve cell or a muscle cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. “De-differentiation” refers to the process by which a cell reverts to a less specialized (or committed) position within the lineage of a cell. As used herein, the lineage of a cell defines the heredity of the cell, i.e., which cells it came from and to what cells it can give rise. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

“Markers”, as used herein, are nucleic acid or polypeptide molecules that are differentially expressed in a cell of interest. In this context, differential expression means an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive for” a specific marker or “positive” when the specific marker is sufficiently detected in the cell. Similarly, the cell is “negative for” a specific marker, or “negative” when the specific marker is not sufficiently detected in the cell. In particular, positive by FACS is usually greater than 2%, whereas the negative threshold by FACS is usually less than 1%. Positive by PCR is usually less than 34 cycles (Cts); whereas negative by PCR is usually more than 34.5 cycles.

In attempts to replicate the differentiation of pluripotent stem cells into functional pancreatic endocrine cells in static in vitro cell cultures, the differentiation process is often viewed as progressing through a number of consecutive stages. In particular, the differentiation process is commonly viewed as progressing through six stages. In this step-wise progression, “Stage 1” refers to the first step in the differentiation process, the differentiation of pluripotent stem cells into cells expressing markers characteristic of definitive endoderm cells (hereinafter referred to alternatively as “Stage 1 cells”). “Stage 2” refers to the second step, the differentiation of cells expressing markers characteristic of definitive endoderm cells into cells expressing markers characteristic of gut tube cells (hereinafter referred to alternatively as “Stage 2 cells”). “Stage 3” refers to the third step, the differentiation of cells expressing markers characteristic of gut tube cells into cells expressing markers characteristic of foregut endoderm cells (hereinafter referred to alternatively as “Stage 3 cells”). “Stage 4” refers to the fourth step, the differentiation of cells expressing markers characteristic of foregut endoderm cells into cells expressing markers characteristic of pancreatic foregut precursor cells (hereinafter referred to alternatively as “Stage 4 cells”). “Stage 5” refers to the fifth step, the differentiation of cells expressing markers characteristic of pancreatic foregut precursor cells into cells expressing markers characteristic of pancreatic endoderm cells and/or pancreatic endocrine precursor cells (hereinafter referred to collectively as “pancreatic endoderm/endocrine precursor cells” or alternatively as “Stage 5 cells”). “Stage 6” refers to the differentiation of cells expressing markers characteristic of pancreatic endoderm/endocrine precursor cells into cells expressing markers characteristic of pancreatic endocrine cells (hereinafter referred to alternatively as “Stage 6 cells”).

However, it should be noted that not all cells in a particular population progress through these stages at the same rate. Consequently, it is not uncommon in in vitro cell cultures to detect the presence of cells that have progressed less, or more, down the differentiation pathway than the majority of cells present in the population, particularly at the later differentiation stages. For example, it is not uncommon to see the appearance of markers characteristic of pancreatic endocrine cells during the culture of cells at Stage 5. For purposes of illustrating the present invention, characteristics of the various cell types associated with the above-identified stages are described herein.

“Definitive endoderm cells,” as used herein, refers to cells which bear the characteristics of cells arising from the epiblast during gastrulation and which form the gastrointestinal tract and its derivatives. Definitive endoderm cells express at least one of the following markers: FOXA2 (also known as hepatocyte nuclear factor 3-β (“HNF3β”)), GATA4, SOX17, CXCR4, Brachyury, Cerberus, OTX2, goosecoid, C-Kit, CD99, and MIXL1. Markers characteristic of the definitive endoderm cells include CXCR4, FOXA2 and SOX17. Thus, definitive endoderm cells may be characterized by their expression of CXCR4, FOXA2 and SOX17. In addition, depending on the length of time cells are allowed to remain in Stage 1, an increase in HNF4α may be observed.

“Gut tube cells,” as used herein, refers to cells derived from definitive endoderm that can give rise to all endodermal organs, such as lungs, liver, pancreas, stomach, and intestine. Gut tube cells may be characterized by their substantially increased expression of HNF4α over that expressed by definitive endoderm cells. For example, a ten to forty fold increase in mRNA expression of HNF4α may be observed during Stage 2.

“Foregut endoderm cells,” as used herein, refers to endoderm cells that give rise to the esophagus, lungs, stomach, liver, pancreas, gall bladder, and a portion of the duodenum. Foregut endoderm cells express at least one of the following markers: PDX1, FOXA2, CDX2, SOX2, and HNF4α. Foregut endoderm cells may be characterized by an increase in expression of PDX1 compared to gut tube cells. For example, greater than fifty percent of the cells in Stage 3 cultures typically express PDX1.

“Pancreatic foregut precursor cells,” as used herein, refers to cells that express at least one of the following markers: PDX1, NKX6.1, HNF6, NGN3, SOX9, PAX4, PAX6, ISL1, gastrin, FOXA2, PTF1a, PROX1 and HNF4α. Pancreatic foregut precursor cells may be characterized by being positive for the expression of PDX1, NKX6.1, and SOX9.

“Pancreatic endoderm cells,” as used herein, refers to cells that express at least one of the following markers: PDX1, NKX6.1, HNF1β, PTF1α, HNF6, HNF4α, SOX9, NGN3; gastrin; HB9, or PROX1. Pancreatic endoderm cells may be characterized by their lack of substantial expression of CDX2 or SOX2.

“Pancreatic endocrine precursor cells,” as used herein, refers to pancreatic endoderm cells capable of becoming a pancreatic hormone expressing cell. Pancreatic endocrine precursor cells express at least one of the following markers: NGN3; NKX2.2; NeuroD1; ISL1; PAX4; PAX6; or ARX. Pancreatic endocrine precursor cells may be characterized by their expression of NKX2.2 and NeuroD1.

“Pancreatic endocrine cells,” as used herein, refer to cells capable of expressing at least one of the following hormones: insulin, glucagon, somatostatin, ghrelin, and pancreatic polypeptide. In addition to these hormones, markers characteristic of pancreatic endocrine cells include one or more of NGN3, NeuroD1, ISL1, PDX1, NKX6.1, PAX4, ARX, NKX2.2, and PAX6. Pancreatic endocrine cells expressing markers characteristic of β cells can be characterized by their expression of insulin and at least one of the following transcription factors: PDX1, NKX2.2, NKX6.1, NeuroD1, ISL1, HNF3β, MAFA and PAX6.

Used interchangeably herein are “d1”, “1d”, and “day 1”; “d2”, “2d”, and “day 2”, and so on. These number letter combinations refer to a specific day of incubation in the different stages during the stepwise differentiation protocol of the instant application.

“Glucose” is used herein to refer to dextrose, a sugar commonly found in nature.

“NeuroD1” is used herein to identify a protein expressed in pancreatic endocrine progenitor cells and the gene encoding it.

“LDN-193189” refers to ((6-(4-(2-(piperidin-1-yl)ethoxy)phenyl)-3-(pyridin-4-yl)pyrazolo[1,5-a]pyrimidine, hydrochloride; DM-3189)) a BMP receptor inhibitor available under the trademark STEMOLECULE™ from Stemgent, Inc., Cambridge, MA, USA.

### Characterization, Source, Expansion and Culture of Pluripotent Stem Cells

### A. Characterization of Pluripotent Stem Cells

Pluripotent stem cells may express one or more of the stage-specific embryonic antigens (SSEA) 3 and 4, and markers detectable using Tra-1-60 and Tra-1-81 antibodies (Thomson et al. 1998, Science 282:1145-1147). Differentiation of pluripotent stem cells in vitro results in the loss of Tra-1-60, and Tra-1-81 expression. Undifferentiated pluripotent stem cells typically have Alkaline phosphatase activity, which can be detected by fixing the cells with 4% paraformaldehyde, and then developing with VECTOR® Red as a substrate, as described by the manufacturer (Vector Laboratories, CA, USA). Undifferentiated pluripotent stem cells also typically express OCT4 and TERT, as detected by RT-PCR.

Another desirable phenotype of propagated pluripotent stem cells is a potential to differentiate into cells of all three germinal layers: endoderm, mesoderm, and ectoderm tissues. Pluripotency of stem cells may be confirmed, for example, by injecting cells into severe combined immunodeficiency (“SCID”) mice, fixing the teratomas that form using 4% paraformaldehyde, and then histologically examining for evidence of cell types from these three germ layers. Alternatively, pluripotency may be determined by the creation of embryoid bodies and assessing the embryoid bodies for the presence of markers associated with the three germinal layers.

Propagated pluripotent stem cell lines may be karyotyped using a standard G-banding technique and compared to published karyotypes of the corresponding primate species. It is desirable to obtain cells that have a “normal karyotype,” which means that the cells are euploid, wherein all human chromosomes are present and not noticeably altered.

### B. Sources of Pluripotent Stem Cells

Exemplary types of pluripotent stem cells that may be used include established lines of pluripotent cells, including pre-embryonic tissue (such as, for example, a blastocyst), embryonic tissue, or fetal tissue taken any time during gestation, typically but not necessarily, before approximately 10 to 12 weeks gestation. Non-limiting examples are established lines of human embryonic stem cells or human embryonic germ cells, such as, for example the human embryonic stem cell lines H1, H7, and H9 (WiCell Research Institute, Madison, WI, USA). Cells taken from a pluripotent stem cell population already cultured in the absence of feeder cells are also suitable. Inducible pluripotent cells (IPS), or reprogrammed pluripotent cells, derived from adult somatic cells using forced expression of a number of pluripotent related transcription factors, such as OCT4, NANOG, SOX2, KLF4, and ZFP42 (Annu Rev Genomics Hum Genet 2011, 12:165-185; see also IPS, Cell, 126(4): 663-676) may also be used. The human embryonic stem cells used in the methods of the invention may also be prepared as described by Thomson et al. (U.S. Pat. No. 5,843,780; Science, 1998, 282:1145-1147; Curr Top Dev Biol 1998, 38:133-165; Proc Natl Acad Sci U.S.A. 1995, 92:7844-7848). Mutant human embryonic stem cell lines, such as, BG01v (BresaGen, Athens, Ga.), or cells derived from adult human somatic cells, such as, cells disclosed in Takahashi et al., Cell 131:1-12 (2007) may also be used. In certain embodiments, pluripotent stem cells suitable for use in the present invention may be derived according to the methods described in: Li et al. (Cell Stem Cell 4: 16-19, 2009); Maherali et al. (Cell Stem Cell 1: 55-70, 2007); Stadtfeld et al. (Cell Stem Cell 2: 230-240); Nakagawa et al. (Nature Biotechnol 26: 101-106, 2008); Takahashi et al. (Cell 131: 861-872, 2007); and U.S. Patent App. Pub. No. 2011/0104805. In certain embodiments, the pluripotent stem cells may be of non-embryonic origins. All of these references, patents, and patent applications are herein incorporated by reference in their entirety, in particular, as they pertain to the isolation, culture, expansion and differentiation of pluripotent cells.

### C. Expansion and Culture of Pluripotent Stem Cells

In one embodiment, pluripotent stem cells are typically cultured on a layer of feeder cells that support the pluripotent stem cells in various ways. Alternatively, pluripotent stem cells are cultured in a culture system that is essentially free of feeder cells, but nonetheless supports proliferation of pluripotent stem cells without undergoing substantial differentiation. The growth of pluripotent stem cells in feeder-free culture without differentiation is supported using a medium conditioned by culturing previously with another cell type. Alternatively, the growth of pluripotent stem cells in feeder-free culture without differentiation is supported using a chemically defined medium.

Pluripotent cells may be readily expanded in culture using various feeder layers or by using matrix protein coated vessels. Alternatively, chemically defined surfaces in combination with defined media such as mTesr®1 media (StemCell Technologies, Vancouver, Canada) may be used for routine expansion of the cells. Pluripotent cells may be readily removed from culture plates using enzymatic digestive, mechanical separation, or various calcium chelators such as EDTA (ethylenediaminetetraacetic acid). Alternatively, pluripotent cells may be expanded in suspension in the absence of any matrix proteins or feeder layer.

Many different methods of expanding and culturing pluripotent stem cells may be used in the claimed invention. For example, the methods of the invention may use the methods of Reubinoff et al., Thompson et al., Richard et al. and U.S. Patent App. Pub. No. 2002/0072117. Reubinoff et al. (Nature Biotechnology 18: 399-404 (2000)) and Thompson et al. (Science 282: 1145-1147 (1998)) disclose the culture of pluripotent stem cell lines from human blastocysts using a mouse embryonic fibroblast feeder cell layer. Richards et al. (Stem Cells 21: 546-556, 2003) evaluated a panel of eleven different human adult, fetal, and neonatal feeder cell layers for their ability to support human pluripotent stem cell culture, noting that human embryonic stem cell lines cultured on adult skin fibroblast feeders retain human embryonic stem cell morphology and remain pluripotent. U.S. Patent App. Pub. No. 2002/0072117 discloses cell lines that produce media that support the growth of primate pluripotent stem cells in feeder-free culture. The cell lines employed are mesenchymal and fibroblast-like cell lines obtained from embryonic tissue or differentiated from embryonic stem cells. U.S. Patent App. Pub. No. 2002/072117 also discloses the use of the cell lines as a primary feeder cell layer.

Other suitable methods of expanding and culturing pluripotent stem cells are disclosed, for example, in Wang et al., Stojkovic et al., Miyamoto et al. and Amit et al. Wang et al. (Stem Cells 23: 1221-1227, 2005) disclose methods for the long-term growth of human pluripotent stem cells on feeder cell layers derived from human embryonic stem cells. Stojkovic et al. (Stem Cells 2005 23: 306-314, 2005) disclose a feeder cell system derived from the spontaneous differentiation of human embryonic stem cells. Miyamoto et al. (Stem Cells 22: 433-440, 2004) disclose a source of feeder cells obtained from human placenta. Amit et al. (Biol. Reprod 68: 2150-2156, 2003) disclose a feeder cell layer derived from human foreskin.

In another embodiment, suitable methods of expanding and culturing pluripotent stem cells are disclosed, for example, in Inzunza et al., U.S. Pat. No. 6,642,048, WO 2005/014799, Xu et al. and U.S. Pub App. No 2007/0010011. Inzunza et al. (Stem Cells 23: 544-549, 2005) disclose a feeder cell layer from human postnatal foreskin fibroblasts. U.S. Pat. No. 6,642,048 discloses media that support the growth of primate pluripotent stem cells in feeder-free culture, and cell lines useful for production of such media. U.S. Pat. No. 6,642,048 reports mesenchymal and fibroblast-like cell lines obtained from embryonic tissue or differentiated from embryonic stem cells; as well as methods for deriving such cell lines, processing media and growing stem cells using such media. WO 2005/014799 discloses a conditioned medium for the maintenance, proliferation, and differentiation of mammalian cells. WO 2005/014799 reports that the culture medium produced via the disclosure is conditioned by the cell secretion activity of murine cells; in particular, those differentiated and immortalized transgenic hepatocytes, named MMH (Met Murine Hepatocyte). Xu et al. (Stem Cells 22:972-980, 2004) discloses a conditioned medium obtained from human embryonic stem cell derivatives that have been genetically modified to over express human telomerase reverse transcriptase. U.S. Pub App. No 2007/0010011 discloses a chemically defined culture medium for the maintenance of pluripotent stem cells.

An alternative culture system employs serum-free medium supplemented with growth factors capable of promoting the proliferation of embryonic stem cells. Examples of such culture systems include, but are not limited, to Cheon et al., Levenstein et al. and U.S. Pub App. No. 2005/0148070. Cheon et al. (BioReprod DOI:10.1095/biolreprod.105.046870, Oct. 19, 2005) disclose a feeder-free, serum-free culture system in which embryonic stem cells are maintained in unconditioned serum replacement (SR) medium supplemented with different growth factors capable of triggering embryonic stem cell self-renewal. Levenstein et al. (Stem Cells 24: 568-574, 2006) disclose methods for the long-term culture of human embryonic stem cells in the absence of fibroblasts or conditioned medium, using media supplemented with bFGF. U.S. Pub App. No. 2005/0148070 discloses a method of culturing human embryonic stem cells in defined media without serum and without fibroblast feeder cells, the method comprising: culturing the stem cells in a culture medium containing albumin, amino acids, vitamins, minerals, at least one transferrin or transferrin substitute, at least one insulin or insulin substitute, the culture medium essentially free of mammalian fetal serum and containing at least about 100 ng/ml of a fibroblast growth factor capable of activating a fibroblast growth factor signaling receptor, wherein the growth factor is supplied from a source other than just a fibroblast feeder layer, the medium supported the proliferation of stem cells in an undifferentiated state without feeder cells or conditioned medium.

Other suitable methods of culturing and expanding pluripotent stem cells are disclosed in U.S. Patent App. Pub. No.2005/0233446, U.S. Pat. No. 6,800,480, U.S. Patent App. Pub. No.2005/0244962 and WO 2005/065354. U.S. Patent App. Pub. No.2005/0233446 discloses a defined media useful in culturing stem cells, including undifferentiated primate primordial stem cells. In solution, the media is substantially isotonic as compared to the stem cells being cultured. In a given culture, the particular medium comprises a base medium and an amount of each of bFGF, insulin, and ascorbic acid necessary to support substantially undifferentiated growth of the primordial stem cells. U.S. Pat. No. 6,800,480 reports that a cell culture medium for growing primate-derived primordial stem cells in a substantially undifferentiated state is provided which includes a low osmotic pressure, low endotoxin basic medium that is effective to support the growth of primate-derived primordial stem cells. The disclosure of the U.S. Pat. No. 6,800,480 further reports that the basic medium is combined with a nutrient serum effective to support the growth of primate-derived primordial stem cells and a substrate selected from the group consisting of feeder cells and an extracellular matrix component derived from feeder cells. This medium is further noted to include non-essential amino acids, an anti-oxidant, and a first growth factor selected from the group consisting of nucleosides and a pyruvate salt. U.S. Patent App. Pub. No. 2005/0244962 reports that one aspect of the disclosure provides a method of culturing primate embryonic stem cells and that the stem cells in culture are essentially free of mammalian fetal serum (preferably also essentially free of any animal serum) and in the presence of fibroblast growth factor that is supplied from a source other than just a fibroblast feeder layer.

WO 2005/065354 discloses a defined, isotonic culture medium that is essentially feeder-free and serum-free, comprising: a basal medium; bFGF; insulin; and ascorbic acid. Furthermore, WO 2005/086845 discloses a method for maintenance of an undifferentiated stem cell, said method comprising exposing a stem cell to a member of the transforming growth factor-β (TGF-β) family of proteins, a member of the fibroblast growth factor (FGF) family of proteins, or nicotinamide (NIC) in an amount sufficient to maintain the cell in an undifferentiated state for a sufficient amount of time to achieve a desired result.

The pluripotent stem cells may be plated onto a suitable culture substrate. In one embodiment, the suitable culture substrate is an extracellular matrix component, such as those derived from basement membrane or that may form part of adhesion molecule receptor-ligand couplings. In one embodiment, the suitable culture substrate is MATRIGEL™ (Becton Dickenson). MATRIGEL™ is a soluble preparation from Engelbreth-Holm Swarm tumor cells that gels at room temperature to form a reconstituted basement membrane.

Other extracellular matrix components and component mixtures are suitable as an alternative. Depending on the cell type being proliferated, this may include laminin, fibronectin, proteoglycan, entactin, heparan sulfate, and the like, alone or in various combinations.

The pluripotent stem cells may be plated onto the substrate in a suitable distribution and in the presence of a medium, which promotes cell survival, propagation, and retention of the desirable characteristics. All these characteristics benefit from careful attention to the seeding distribution and can readily be determined by one of skill in the art. Suitable culture media may be made, for example, from the following components: Dulbecco's modified Eagle's medium (DMEM), sold under the trademark Gibco™ (part #11965-092) by Life Technologies Corporation, Grand Island, NY; Knockout Dulbecco's modified Eagle's medium (KO DMEM), sold under the trademark Gibco™ (part #10829-018) by Life Technologies Corporation, Grand Island, NY; Ham's F12/50% DMEM basal medium; 200 mM L-glutamine, sold under the trademark Gibco™ (part #15039-027) by Life Technologies Corporation, Grand Island, NY; non-essential amino acid solution, sold under the trademark Gibco™ (part #11140-050) by Life Technologies Corporation, Grand Island, NY; β-mercaptoethanol, (part #M7522) sold by Sigma-Aldrich, Company, LLC, Saint Louis, MO; and human recombinant basic fibroblast growth factor (bFGF), sold under the trademark Gibco™ (part #13256-029) by Life Technologies Corporation, Grand Island, NY.

### Differentiation of Pluripotent Stem Cells

As pluripotent cells differentiate towards β cells, they differentiate through various stages each of which may be characterized by the presence or absence of particular markers. Differentiation of the cells into these stages is achieved by the specific culturing conditions including the presence or lack of certain factors added to the culture media. In general, this differentiation may involve differentiation of pluripotent stem cells into definitive endoderm cells. These definitive endoderm cells may then be further differentiated into gut tube cells, which in turn may then be differentiated into foregut endoderm cells. Foregut endoderm cells may be differentiated into pancreatic foregut precursor cells which can, in turn, differentiate into pancreatic endoderm cells, pancreatic endocrine precursor cells or both. These cells may then be differentiated into pancreatic hormone producing cells (such as β cells).

This invention provides for staged differentiation of pluripotent stem cells toward pancreatic endocrine cells using a thyroid hormone (such as T3, analogues of T3, T4, analogues of T4 or combinations thereof (collectively referred to hereinafter as “T3/T4”)) and an ALK5 inhibitor. This invention also provides for staged differentiation of pluripotent stem cells toward pancreatic endocrine cells using a thyroid hormone (such as T3/T4) or an ALK5 inhibitor. Suitable thyroid hormone analogues may include: GC-1 (Sobertirome) available from R & D Systems, Inc. Catalogue #4554; DITPA (3,5-diiodothyropropionic acid); KB-141, discussed in J. Steroid Biochem. Mol. Biol. 2008, 111: 262-267 and Proc. Natl. Acad. Sci. US 2003, 100: 10067-10072; MB07344, discussed in Proc. Natl. Acad. Sci. US 2007, 104: 15490-15495; T0681, discussed in PLoS One, 2010, 5e8722 and J. Lipid Res. 2009, 50: 938-944; and GC-24, discussed in PLoS One, 2010 e8722 and Endocr. Pract. 2012, 18(6): 954-964, the disclosures of which are incorporated herein in their entirety. Useful ALK5 inhibitors include: ALK5 inhibitor II (Enzo, Farmingdale, NY); ALK5i (Axxora, San Diego, CA); SD208 (R & D systems (MN)); TGF-B inhibitor SB431542 (Xcess Biosciences (San Diego, CA)); ITD-1 (Xcess Biosciences (San Diego, CA)); LY2109761 (Xcess Biosciences (San Diego, CA)); A83-01 (Xcess Biosciences (San Diego, CA)); LY2157299 (Xcess Biosciences (San Diego, CA)); TGF-β receptor inh V (EMD Chemicals, Gibstown, NJ); TGF-β receptor inh I (EMD Chemicals, Gibstown, NJ); TGF-β receptor inh IV (EMD Chemicals, Gibstown, NJ); TGF-β receptor inh VII (EMD Chemicals, Gibstown, NJ); TGF-β receptor inh VIII (EMD Chemicals, Gibstown, NJ); TGF-β receptor inh II (EMD Chemicals, Gibstown, NJ); TGF-β receptor inh VI (EMD Chemicals, Gibstown, NJ); TGF-β receptor inh III (EMD Chemicals, Gibstown, NJ).

### Differentiation of Pluripotent Stem Cells into Cells Expressing Markers Characteristic of Pancreatic Endocrine Cells

Characteristics of pluripotent stem cells are well known to those skilled in the art, and additional characteristics of pluripotent stem cells continue to be identified. Pluripotent stem cell markers include, for example, the expression of one or more of the following: ABCG2; cripto; FOXD3; CONNEXIN43; CONNEXIN45; OCT4; SOX2; NANOG; hTERT; UTF1; ZFP42; SSEA-3; SSEA-4; TRA-1-60; and TRA-1-81.

Exemplary pluripotent stem cells include the human embryonic stem cell line H9 (NIH code: WA09), the human embryonic stem cell line H1 (NIH code: WA01), the human embryonic stem cell line H7 (NIH code: WA07), and the human embryonic stem cell line SA002 (Cellartis, Sweden). Also suitable are cells that express at least one of the following markers characteristic of pluripotent cells: ABCG2, cripto, CD9, FOXD3, CONNEXIN43, CONNEXIN45, OCT4, SOX2, NANOG, hTERT, UTF1, ZFP42, SSEA-3, SSEA-4, TRA-1-60, and TRA-1-81.

Also suitable for use in the present invention is a cell that expresses at least one of the markers characteristic of the definitive endoderm lineage. In one embodiment of the invention, a cell expressing markers characteristic of the definitive endoderm lineage is a primitive streak precursor cell. In an alternate embodiment, a cell expressing markers characteristic of the definitive endoderm lineage is a mesendoderm cell. In an alternate embodiment, a cell expressing markers characteristic of the definitive endoderm lineage is a definitive endoderm cell.

Also suitable for use in the present invention is a cell that expresses at least one of the markers characteristic of the pancreatic endoderm lineage. In one embodiment of the present invention, a cell expressing markers characteristic of the pancreatic endoderm lineage is a pancreatic endoderm cell in which the expression of PDX1 and NKX6.1 are substantially higher than the expression of CDX2 and SOX2. Particularly useful are cells in which the expression of PDX1 and NKX6.1 is at least two-fold higher than the expression of CDX2 or SOX2.

In one embodiment, pancreatic endocrine cells capable of expressing at least one of the following hormones: insulin, glucagon, somatostatin, and pancreatic polypeptide are generated. Suitable for use in the present invention is a precursor cell that expresses at least one of the markers characteristic of the pancreatic endocrine lineage. In one embodiment of the present invention, a cell expressing markers characteristic of the pancreatic endocrine lineage is a pancreatic endocrine cell. In a preferred embodiment, the pancreatic endocrine cell is an insulin-producing β cell.

In certain embodiments of the invention, to arrive at the cells expressing markers characteristic of pancreatic endocrine cells, a protocol starting with pluripotent stem cells or inducible pluripotent cells, preferably pluripotent stem cells, is employed. This protocol includes the following stages.
Stage 1: Pluripotent stem cells, such as embryonic stem cells obtained for cell culture lines, are treated with appropriate factors to induce differentiation into cells expressing markers characteristic of definitive endoderm cells.
Stage 2: Cells resulting from Stage 1 are treated with appropriate factors to induce further differentiation into cells expressing markers characteristic of gut tube cells.
Stage 3: Cells resulting from Stage 2 are treated with appropriate factors to induce further differentiation into cells expressing markers characteristic of foregut endoderm cells.
Stage 4: Cells resulting from Stage 3 are treated with appropriate factors (including in certain embodiments T3/T4) to induce further differentiation into cells expressing markers characteristic of pancreatic foregut precursor cells.
Stage 5: Cells resulting from Stage 4 are treated with appropriate factors (including in certain embodiments: (i) T3/T4; (ii) an ALK5 inhibitor; or (iii) both T3/T4 and an ALK 5 inhibitor) to induce further differentiation into cells expressing markers characteristic of pancreatic endoderm/endocrine precursor cells.
Stage 6: Cells resulting from Stage 5 are treated with appropriate factors (including in certain embodiments T3/T4, an ALK5 inhibitor, or both) to induce further differentiation into cells expressing markers characteristic of pancreatic endocrine cells.

While the invention, in certain embodiments, encompasses differentiating pluripotent stem cells to cells expressing markers characteristic of pancreatic endocrine cells, the invention also encompasses differentiating cells resulting from other intermediate stages towards pancreatic endocrine cells. In particular, the invention encompasses differentiation of cells expressing markers characteristic of pancreatic foregut precursor cells into cells expressing markers characteristic of pancreatic endocrine cells. Moreover, although the process is described in discrete stages, the treatment, as well as the progress of the cells through the differentiation process, may be sequential or continuous.

Methods for assessing expression of protein and nucleic acid markers in cultured or isolated cells are standard in the art. These methods include RT-PCR, Northern blots, in situ hybridization (see, e.g., Current Protocols in Molecular Biology (Ausubel et al., eds. 2001 supplement)), and immunoassays (such as immunohistochemical analysis of sectioned material), Western blotting, and for markers that are accessible in intact cells, FACS (see, e.g., Harlow and Lane, Using Antibodies: A Laboratory Manual, New York: Cold Spring Harbor Laboratory Press (1998)). Further, the efficiency of differentiation may be determined by exposing a treated cell population to an agent (such as an antibody) that specifically recognizes a protein marker expressed by cells expressing markers characteristic of the cell type of interest.

The differentiated cells may also be further purified. For example, after treating pluripotent stem cells with the methods of the present invention, the differentiated cells may be purified by exposing a treated cell population to an agent (such as an antibody) that specifically recognizes a protein marker characteristically expressed by the differentiated cells being purified.
Stage 1: Differentiation of pluripotent stem cells into cells expressing markers characteristic of definitive endoderm cells

Pluripotent stem cells may be differentiated into cells expressing markers characteristic of definitive endoderm cells by any suitable method known in the art, or by any method proposed in this invention. Suitable methods of differentiating pluripotent stem cells into cells expressing markers characteristic of definitive endoderm cells are disclosed in: U.S. Patent App. Pub. No. 2007/0254359; U.S. Patent App. Pub. No. 2009/0170198; U.S. Patent App. Pub. No. 2009/0170198; U.S. Patent App. Pub. No. 2011/0091971; U.S. Patent App. Pub. No. 2010/0015711; U.S. Patent App. Pub. No. 2010/0015711; U.S. Patent App. Pub. No. 2012/0190111; U.S. Patent App. Pub. No. 2012/0190112; U.S. Patent App. Pub. No. 2012/0196365; U.S. Patent App. Pub. No. 20100015711; U.S. Patent App. Pub. No. 2012/0190111; U.S. Patent App. Pub. No. 2012/0190112; U.S. Patent App. Pub. No. 2012/0196365; U.S. Patent App. Pub. No. 20100015711; U.S. Patent App. Pub. No. 2012/0190111; U.S. Patent App. Pub. No. 2012/0190112; U.S. Patent App. Pub. No. 2012/0196365; U.S. Provisional Patent Application No. 61/076,900; U.S. Provisional Patent Application No. 61/076,908; and U.S. Provisional Patent Application No. 61/076,915, which are incorporated by reference in their entireties as they relate to pluripotent stem cells and to the differentiation of pluripotent stem cells into cells expressing markers characteristic of definitive endoderm cells.

In one embodiment of the invention, pluripotent stem cells are treated with a medium supplemented with activin A and Wnt3A to result in the generation of cells expressing markers characteristic of definitive endoderm cells. Treatment may involve contacting pluripotent stem cells with a medium containing about 50 ng/ml to about 150 ng/ml, alternatively about 75 ng/ml to about 125 ng/ml, alternatively about 100 ng/ml of activin A. The treatment may also involve contacting the cells with about 10 ng/ml to about 50 ng/ml, alternatively about 15 ng/ml to about 30 ng/ml, alternatively about 20 ng/ml of Wnt3A. The pluripotent cells may be cultured for approximately two to five days, preferably about two to three days, to facilitate their differentiation into cells expressing markers characteristic of definitive endoderm cells. In one embodiment, the pluripotent cells are cultured in the presence of activin A and Wnt3A for one day, followed by culturing in the presence of activin A (without Wnt3A being present).

In another embodiment of the invention, pluripotent stem cells are treated with a medium supplemented with growth differentiation factor 8 (“GDF8”) and a glycogen synthase kinase-3β (“GSK3B”) inhibitor (such as the cyclic aniline-pyridinotriazine compounds disclosed in U.S. Patent App. Pub. No. 2010/0015711; incorporated herein by reference in its entirety) to induce differentiation into cells expressing markers characteristic of definitive endoderm cells. A preferred GSK3β inhibitor is (14-Prop-2-en-1-yl-3,5,7,14,17,23,27-heptaazatetracyclo [19.3.1.1˜2,6˜0.1˜8,12˜]heptacosa-1(25),2(27),3,5,8(26),9,11,21,23-nonaen-16-one, referred to herein as “MCX Compound”. Treatment may involve contacting pluripotent stem cells with a medium supplemented with about 50 ng/ml to about 150 ng/ml, alternatively about 75 ng/ml to about 125 ng/ml, alternatively about 100 ng/ml of GDF8. The treatment may also involve contacting the cells with about 0.1 to 5 μM, alternatively about 0.5 to about 2.5 μM, preferable about 1 μM of MCX compound. The pluripotent cells may be cultured for approximately two to five days, preferably about three to four days, to facilitate their differentiation into definitive endoderm cells. In one embodiment, the pluripotent cells are cultured in the presence of GDF8 and MCX compound for one day, followed by culturing in the presence of GDF8 and a lower concentration of MCX compound for one day, followed by culturing in the presence of GDF8 for one day in the absence of the MCX compound. In particular, the cells may be cultured in the presence of GDF8 and about 1 μM of MCX compound for one day, followed by culturing in the presence of GDF8 and about 0.1 μM MCX compound for one day, followed by culturing in the presence of GDF8 for one day in the absence of the MCX compound. In an alternate embodiment, the cells may be cultured in the presence of GDF8 and about 1 μM of MCX compound for one day, followed by culturing in the presence of GDF8 and about 0.1 μM MCX compound for one day.

Generation of cells expressing markers characteristic of definitive endoderm cells may be determined by testing for the presence of the markers before and after following a particular protocol. Pluripotent stem cells typically do not express such markers. Thus, differentiation of pluripotent cells can be detected when the cells begin to express markers characteristic of definitive endoderm cells.
Stage 2: Differentiation of cells expressing markers characteristic of definitive endoderm cells into cells expressing markers characteristic of gut tube cells

The cells expressing markers characteristic of definitive endoderm cells may be further differentiated into cells expressing markers characteristic of gut tube cells. In one embodiment, the formation of cells expressing markers characteristic of gut tube cells includes culturing cells expressing markers characteristic of definitive endoderm cells with a medium containing fibroblast growth factor (“FGF”) 7 or FGF10 to differentiate these cells. For example, the culture medium may include from about 25 ng/ml to about 75 ng/ml, alternatively from about 30 ng/ml to about 60 ng/ml, alternatively about 50 ng/ml of FGF7 or FGF10, preferably FGF7. The cells may be cultured under these conditions for about two to three days, preferably about two days.

In another embodiment, differentiation into cells expressing markers characteristic of gut tube cells includes culturing cells expressing markers characteristic of definitive endoderm cells with FGF7 or FGF10, and ascorbic acid (vitamin C). The culture medium may include from about 0.1 mM to about 0.5 mM ascorbic acid, alternatively from about 0.2 mM to about 0.4 mM, alternatively about 0.25 mM of ascorbic acid. The culture medium may also include from about 10 ng/ml to about 35 ng/ml, alternatively from about 15 ng/ml to about 30 ng/ml, alternatively about 25 ng/ml of FGF7 or FGF10, preferably FGF7. For example, the culture medium may include about 0.25 mM of ascorbic acid and about 25 ng/ml of FGF-7. In one embodiment, cells expressing markers characteristic of definitive endoderm cells are treated for 2 days with FGF7 and ascorbic acid.
Stage 3: Differentiation of cells expressing markers characteristic of gut tube cells into cells expressing markers characteristic of foregut endoderm cells

Cells expressing markers characteristic of gut tube cells may be further differentiated into cells expressing markers characteristic of foregut endoderm cells. In one embodiment, Stage 2 cells are further differentiated into Stage 3 cells by culturing these cells in a culture medium supplemented with a Smoothened (“SMO”) receptor inhibitor (such as cyclopamine or MRT10 (N-[[[3-benzoylamino)phenyl]amino]thioxomethyl]-3,4,5-trimethoxybenzamide)) or a Sonic Hedgehog (“SHH”) signaling pathway antagonist (such as Smoothened Antogonist 1 (“SANT-1”) ((E)-4-benzyl-N-((3,5-dimethyl-1-phenyl-1H-pyrazol-4-yl) methylene-piperazin-1-amine)), Hedgehog Pathway Inhibitor 1 (“HPI-1”) (2-methoxyethyl 1,4,5,6,7,8-hexahydro-4-(3-hydroxyphenyl)-7-(2-methoxyphenyl)-2-methyl-5-oxo-3-quinolinecarboxylate), retinoic acid and Noggin. Alternatively, the medium may be supplemented with a SMO inhibitor, SHH signaling pathway antagonist, retinoic acid and Noggin. The cells may be cultured for approximately two to four days, preferably about two days. In one embodiment, the medium is supplemented with from about 0.1 μM to about 0.3 μM of SANT-1, from about 0.5 μM to about 3 μM of retinoic acid and from about 75 ng/ml to about 125 ng/ml of Noggin. In another embodiment, the medium is supplemented with about 0.25 μM of SANT-1, about 2 μM of retinoic acid and about 100 ng/ml of Noggin.

In an alternate embodiment, Stage 2 cells are further differentiated into Stage 3 cells by treating the Stage 2 cells with a medium supplemented with FGF7 or FGF10, retinoic acid, a SMO inhibitor (such as MRT10 or cyclopamine) or SHH signaling pathway antagonist (such as SANT-1 or HPI-1), a protein Kinase C (“PKC”) activator (such as ((2S,5S)-(E,E)-8-(5-(4-(Trifluoromethyl)phenyl)-2,4-pentadienoylamino)benzolactam) (“TPB”); EMD Chemicals Inc., Gibbstown NJ), phorbol-12, 13-dibutyrate (“PDBu”), phorbol-12-myristate-13-acetate (“PMA”) or indolactam V (“ILV”), a bone morphogenic protein (“BMP”) inhibitor (such as LDN-193189, Noggin or Chordin), and ascorbic acid. In another embodiment, the medium may be supplemented with FGF7 or FGF10, retinoic acid, a SMO inhibitor, a SHH signaling pathway antagonist (such as SANT-1), a PKC activator (such as TPB), a BMP inhibitor (such as LDN-193189), and ascorbic acid. The cells may be cultured in the presence of these growth factors, small molecule agonists, and antagonists for about two to three days.

In one embodiment, the medium is supplemented with from about 15 ng/ml to about 35 ng/ml of FGF7, from about 0.5 μM to about 2 μM of retinoic acid, from about 0.1 μM to about 0.4 μM of SANT-1, from about 100 nM to about 300 nM of TPB, from about 50 nM to about 200 nM of LDN-193189, and from about 0.15 mM to about 0.35 mM of ascorbic acid. In another embodiment, the medium is supplemented with about 25 ng/ml of FGF7, about 1 μM of retinoic acid, about 0.25 μM of SANT-1, about 200 nM of TPB, about 100 nM of LDN-193189, and about 0.25 mM of ascorbic acid.
Stages 4 to 6: Differentiation of cells expressing markers characteristic of foregut endoderm cells into cells expressing markers characteristic of pancreatic endoderm cells by treatment with culture media supplemented with thyroid hormones T3/T4 or ALK5 inhibitor, or both T3/T4 and ALK5 inhibitor.

This invention provides for the further differentiation of cells expressing markers characteristic of foregut endoderm cells by treatment with culture media supplemented with thyroid hormone T3/T4, or an ALK5 inhibitor, or both T3/T4 and an ALK5 inhibitor. In some embodiments, the invention provides for further differentiation of such cells in Stage 4 to Stage 6 by treatment with culture media supplemented with (a) T3, (b) an ALK5 inhibitor or (c) T3 and an ALK5 inhibitor at one or more of these stages.

In one embodiment, the present invention provides a method for producing cells expressing markers characteristic of pancreatic endocrine cells from pluripotent stem cells comprising:
a. culturing pluripotent stem cells;
b. differentiating the pluripotent stem cells into cells expressing markers characteristic of foregut endoderm cells; and
c. differentiating the cells expressing markers characteristic of foregut endoderm cells into cells expressing markers characteristic of pancreatic endocrine cells by treatment with a medium supplemented with (i) T3/T4, (ii) an ALK5 inhibitor, or (iii) both T3/T4 and an ALK5 inhibitor.

In one embodiment, the cells expressing markers characteristic of pancreatic endocrine cells are β cells. In another embodiment, the resulting cells are positive for NKX6.1, PDX1, and HB-9. The method may enhance the number of HB9 positive cells in NKX6.1 positive pancreatic endoderm precursor cells. The method may also decrease expression of NKX2.2 or SOX2, or both, as well as albumin expression. The method may also provide cells expressing markers characteristic of pancreatic endocrine cells, including β cells, by culturing cells expressing markers characteristic of pancreatic endoderm/endocrine cells in a medium supplemented with T3/T4. The methods of producing cells expressing markers characteristic of pancreatic endocrine cells from pluripotent stem cells may employ the culture conditions shown in the Tables I to III, or described herein. In one embodiment, the ALK 5 inhibitor is SD208 (2-(5-Chloro-2-fluorophenyl)pteridin-4-yl]pyridin-4-yl-amine). In another embodiment, ALK5 inhibitor II ((2-(3-(6-Methylpyridin-2-yl)-1H-pyrazol-4-yl)-1,5-naphthyridine), ALX-270-445, ENZO, Farmingdale, NY,) can also be used.

Treatment of cells in Stages 4 to 6 with culture media supplemented with T3/T4, an ALK5 inhibitor, or both provides for several advantages. For example, the addition of the thyroid hormones at Stage 4 to Stage 6 significantly downregulates glucagon, somatostatin, and ghrelin while moderately increasing insulin expression at Stage 5. The addition of T3/T4 at Stages 4 to 6 also appears to significantly decrease expression of NKX2.2 while not impacting NKX6.1 and PDX1 expression. Furthermore, T3/T4 addition at Stages 4 to 6 suppresses SOX2 (stomach marker) and albumin (liver marker) expression while not affecting CDX2 (intestine marker) expression. Moreover, compared to an untreated control, treatment with T3 at Stage 4 increases the number of HB9 positive cells at Stage 6. Furthermore, T3 treatment resulted in an increased number of NKX6.1 positive cells that express HB9. Prolonged exposure to both an ALK5 inhibitor and T3/T4 appears to significantly enhance expression of HB9 while maintaining robust expression of NKX6.1. The inclusion of T3/T4 in a culture medium, appears, in a dose dependent manner, to significantly enhance the number of HB9 positive cells in the NKX6.1 positive pancreatic endoderm precursor cells.

Accordingly, in certain embodiments, the invention provides for methods of down-regulating glucagon, somatostatin and ghrelin in the differentiated cells provided in Stage 4 to Stage 6 by treatment with a medium supplemented with at least thyroid hormones T3/T4. Moreover, the invention also provides for methods of decreasing NKX 2.2 expression in the differentiated cells provided in Stage 4 to Stage 6 that express NKX6.1 and PDX1 by treatment with a medium supplemented with at least thyroid hormones T3/T4. In addition, the invention provides methods for increasing NKX6.1 positive cells expressing HB9 by culturing in a medium with thyroid hormones T3/T4 and optionally an ALK5 inhibitor. In certain embodiments, the methods use the culture conditions shown in Tables I-III.

One embodiment of the invention is a method of forming cells expressing markers characteristic of β cells comprising differentiating cells expressing markers characteristic of the foregut endoderm into cells expressing markers characteristic of β cells by treatment with media supplemented with thyroid hormones T3/T4, an ALK5 inhibitor, or both (such as T3 and an ALK5 inhibitor). The resulting cells are positive for NKX6.1, PDX1, and Hb-9. The method may be used to enhance the number of HB9 positive cells in NKX6.1 positive pancreatic endoderm precursor cells. The method may also be used to decrease expression of NKX2.2. Additionally, the method may be used to suppress SOX2 and albumin expression. The thyroid hormone may be T3. The method may also be used to enhance HB9 expression when compared to cells that are not cultured with a medium supplemented with T3 and an ALK5 inhibitor. Furthermore, the method comprises formation of cells expressing markers characteristic of B cells by culturing cells expressing cells markers characteristic of pancreatic endoderm/endocrine precursor cells in a medium supplemented with T3/T4. The method may employ the culture conditions shown in Tables I-III or described herein.

Yet another embodiment of the invention is a method of down-regulating glucagon, somatostatin and ghrelin in cells expressing markers characteristic of pancreatic foregut precursor cells, cells expressing markers characteristic of pancreatic endoderm/endocrine precursor cells or cells expressing markers characteristic of pancreatic endocrine cells by culturing the cells in a medium supplemented with T3/T4 and an ALK5 inhibitor. The medium may be further supplemented with a SMO inhibitor, a SHH signaling pathway antagonist (such as SANT-1), retinoic acid, and ascorbic acid. Alternatively, the medium may be further supplemented with a SMO inhibitor or a SHH signaling pathway antagonist, retinoic acid, and ascorbic acid. The medium, especially when used at Stage 4, may preferably be supplemented with FGF7. In certain embodiments, the method employs the culture conditions shown in Tables I-III or described herein.

Specifically, in certain embodiments, the cells may be treated in Stage 4 to Stage 6 (i.e. in Stage 4 and Stage 5 and Stage 6, or in Stage 4 and Stage 5, or in Stage 5 and Stage 6, or in Stage 4 and Stage 6) as outlined in Table I below, which shows exemplary culture conditions suitable for use in the methods of the invention. In certain embodiments, any one of the treatments at one stage (e.g. Stage 4) may be combined with any one of the treatments at another stage (e.g. Stage 5).

In an alternate embodiment, the present invention provides an in vitro cell culture for differentiating cells derived from pluripotent stem cells into cells expressing markers characteristic of pancreatic endocrine β cells, as well as PDX1, NKX6.1 and HB9. The cell culture comprises a culture vessel, differentiation medium, and a population of differentiated cells derived from pluripotent stem cells. The cell culture provides a population of differentiated cells wherein at least ten percent of the differentiated cells express PDX1, NKX6.1 and HB9. Media useful in the cell culture are set forth in Tables I-III, and preferably contain T3/T4, or an ALK5 inhibitor, or both.

TABLE I
Exemplary Culture Conditions suitable for use in the methods of the invention

| | Stage 4 | Stage 5 | Stage 6 |
|---|---|---|---|
| Treatment of | Stage 3 cells | Stage 4 cells | Stage 5 cells |
| with at least | T3 | ALK5 inhibitor + T3 | T3 |
| | T3 | ALK5 inhibitor + T3 | ALK5 inhibitor + T3 |
| | T3 | T3 | T3 |
| Other optional | One or more of: | One or more of: | One or more of: |
| components | SANT-1 | SANT-1 | SANT-1 |
| | Retinoic Acid | Retinoic Acid | Retinoic Acid |
| | Ascorbic Acid | Ascorbic Acid | Ascorbic Acid |
| | FGF7 | | |
| | BMP Receptor Inhibitor | | |
| | (e.g. LDN-193189) | | |
| | PKC activator (e.g. TPB) | | |
| Duration of | Approximately 2-4 days, | Approximately 2-4 days, | Approximately 2-4 days, |
| Treatment | preferably about 3 days | preferably about 3 days | preferably about 3 days |

While T3 is generally preferred, other thyroid hormones may be used in place of T3. In particular, T4 may be used in place of T3 as well as suitable analogs of T3 and T4. Suitable thyroid hormone analogues may include: GC-1 (Sobertirome) available from R & D Systems, Inc. Catalogue #4554; DITPA (3,5-diiodothyropropionic acid); KB-141, discussed in J. Steroid Biochem. Mol. Biol. 2008, 111:262-267 and Proc. Natl. Acad. Sci. US 2003, 100: 10067-10072; MB07344, discussed in Proc. Natl. Acad. Sci. US 2007, 104: 15490-15495; T0681, discussed in PLOS One, 2010, 5e8722 and J. Lipid Res. 2009, 50: 938-944; and GC-24, discussed in PLOS One, 2010 e8722 and Endocr. Pract. 2012, 18(6): 954-964, the disclosures of which are incorporated herein in their entirety. The amounts of T3, ALK5 inhibitor, SANT-1, Retinoic Acid, Ascorbic Acid, FGF7, LDN-193189, and TPB may vary in each stage. Exemplary suitable ranges of these components are shown below in Table II.

TABLE II
Exemplary amounts of culture components suitable for use in the methods of the invention

| Component | Exemplary Suitable Amount | Alternatively |
|---|---|---|
| T3 | about 0-1,000 nM | About 1 to about 1000 nM, about 10 to about 900 nM, |
| | | about 100 to about 800 nM, about 200 to about 700 nM, |
| | | about 300 to about 600 nM, about 400 to about 500 nM, |
| | | about 1 to about 500 nM, about 1 to about 100 nM, |
| | | about 100 to about 1000 nM, about 500 to about 1000 nM, |
| | | about 100 nM, about 500 nM, or about 1 μM |
| ALK5 inhibitor | about to 250 nM to about 2 μM | About 300 to about 2000 nM, about 400 to about 2000 nM, |
| | | about 500 to about 2000 nM, about 600 to about 2000 nM, |
| | | about 700 to about 2000 nM, about 800 to about 2000 nM, |
| | | about 1000 to about 2000 nM, about 1500 to about 2000 nM, |
| | | about 250 to about 1000 nM, about 250 to about 500 nM, |
| | | about 300 to about 1000 nM, about 400 to about 1000 nM, |
| | | about 500 to about 1000 nM, about 600 to about 1000 nM, |
| | | about 700 to about 1000 nM, about 800 to about 1000 nM, |
| | | about 100 nM, about 500 nM or about 1 μM |
| SANT-1 | from about 0.1 μM to about 0.3 μM | about 0.25 μM |
| Retinoic Acid | From about 100-2000 nM for stage 3 | From about 200-1800, about 300-1700, about 400-1500, |
| | from about 25 nM to about 150 nM | about 500-1500, about 500-1000 nM for stage 3 |
| | for stages 4, 5, and 6 | about 25 nM to about 100 nM, about 50 nM to about 150 nM, |
| | | about 50 nM to about 100 nM, about 25 nM, about 50 nM, |
| | | or about 100 nM for stages 4, 5, and 6 |
| Ascorbic Acid | from about 0.1 to about 0.4 mM | About 0.1 to about 0.3 mM, about 0.1 to about 0.25 mM, |
| | | about 0.1 to about 0.2 mM, about 0.1 to about 0.15 mM, |
| | | about 0.15 to about 0.4 mM, about 0.2 to about 0.4 mM, |
| | | about 0.25 to about 0.4 mM, about 0.3 to about 0.4 mM, |
| | | about 0.1 mM, about 0.2 mM, about 0.3 mM, about 0.4 mM |
| | | or about 0.25 mM |
| FGF7 | From about 2 to about 35 ng/ml | About 2 to about 30 ng/ml, about 5 to about 25 ng/ml, |
| | | about 10 to about 20 ng/ml, about 2 to about 25 ng/ml, |
| | | about 2 to about 20 ng/ml, about 2 to about 15 ng/ml, |
| | | about 2 to about 10 ng/ml, about 2 to about 5 ng/ml, |
| | | about 5 to about 35 ng/ml, about 10 to about 35 ng/ml, |
| | | about 15 to about 35 ng/ml, about 20 to about 35 ng/ml, |
| | | about 25 to about 35 ng/ml, about 30 to about 35 ng/ml, |
| | | about 2 ng/ml, about 5 ng/ml, about 10 ng/ml, |
| | | about 15 ng/ml, about 20 ng/ml about 25 ng/ml, |
| | | about 30 ng/ml or about 35 ng/ml |
| BMP Receptor Inhibitor | From about 50 to about 150 mM | About 50 to about 140 nM, about 50 to about 130 nM, |
| (e.g. LDN) | | about 50 to about 120 nM, about 50 to about 110 nM, |
| | | about 50 to about 100 nM, about 50 to about 90 nM, |
| | | about 50 to about 80 nM, about 60 to about 150 nM, |
| | | about 70 to about 150 nM, about 80 to about 150 nM, |
| | | about 90 to about 150 nM, about 100 to about 150 nM, |
| | | about 80 to about 120 nM, about 90 to about 110 nM, |
| | | about 50 nM, about 100 nM, or about 150 nM. |
| PKC activator (e.g. TPB) | From about 50 to about 150 mM | About 50 to about 140 nM, about 50 to about 130 nM, |
| | | about 50 to about 120 nM, about 50 to about 110 nM, |
| | | about 50 to about 100 nM, about 50 to about 90 nM, |
| | | about 50 to about 80 nM, about 60 to about 150 nM, |
| | | about 70 to about 150 nM, about 80 to about 150 nM, |
| | | about 90 to about 150 nM, about 100 to about 150 nM, |
| | | about 80 to about 120 nM, about 90 to about 110 nM, |
| | | about 50 nM, about 100 nM, or about 150 nM. |

In one embodiment, the methods of the invention include treating cells expressing markers characteristic of foregut endoderm cells with a medium supplemented with SANT-1, retinoic acid (“RA”), FGF7, LDN-193189, ascorbic acid, and TPB for about two to four days, preferably about three days, to differentiate them into cells expressing markers characteristic of pancreatic foregut precursor cells. In particular, Stage 3 cells may be treated with a medium supplemented with about 0.25 μM SANT-1; about 100 nM RA; about 2 ng/ml FGF7; about 100 nM LDN-193189; and about 0.25 mM ascorbic acid; and about 100 nM TPB for three days. In one embodiment, the medium is further supplemented with T3, such as about 1 μM of T3. In another embodiment, the medium may be supplemented with an ALK5 inhibitor such as about 1 μM of ALK5 inhibitor.

In an alternate embodiment, the methods of the invention include treating cells expressing markers characteristic of pancreatic foregut precursor cells with a medium supplemented with SANT-1, RA, ascorbic acid, and an ALK5 inhibitor for about two to three days to differentiate the cells into cells expressing markers characteristic of pancreatic endoderm/endocrine precursor cells. In certain embodiments, the medium may be further supplemented with T3. In one embodiment, Stage 4 cells are differentiated into Stage 5 cells by treating the cells with a medium supplemented with about 0.25 μM SANT-1, about 50 nM RA, about 0.25 mM ascorbic acid, and about 500 nM ALK5 inhibitor. In another embodiment, the Stage 4 cells are further differentiated into Stage 5 cells by treating the cells with a medium supplemented with about 0.25 μM SANT-1, about 50 nM RA, about 0.25 mM ascorbic acid, about 1 μM ALK5 inhibitor and 0-1000 (e.g. 100) nM T3/T4 for about two to four days, preferably about three days. In one embodiment, Stage 4 cells derived according to embodiments of the invention are utilized and differentiated into Stage 5 cells, while in other embodiments Stage 4 cells derived according to other protocols may be utilized.

In one embodiment of the invention, cells expressing markers characteristic of pancreatic endoderm/endocrine precursor cells are differentiated into cells expressing markers characteristic of pancreatic endocrine cells by treating them with a medium supplemented with SANT-1, RA, ascorbic acid and either (1) T3/T4 or (2) T3/T4 and ALK5 inhibitor for about two to four days, preferably about three days. For example, Stage 5 cells may be differentiated into Stage 6 cells by treatment with a medium supplemented with about 0.25 μM SANT-1, about 50 nM RA, about 0.25 mM ascorbic acid and about 1 μM of T3/T4 for about three days. Alternatively, Stage 5 cells may be differentiated into Stage 6 cells by treatment with a medium supplemented with about 0.25 μM SANT-1, about 50 nM RA, about 0.25 mM ascorbic acid, about 500 nM ALK5 inhibitor and 10 nM T3/T4 for about three days. Alternatively, Stage 5 cells may be differentiated into Stage 6 cells by treatment with a medium supplemented with about 0.25 μM SANT-1, about 50 nM RA, about 0.25 mM ascorbic acid, about 1 μM of ALK5 inhibitor and 0-1000 nM T3/T4 for about three days. The cells may be further cultured in such media as desired, for example, for a total of about 15 days.

In one embodiment, Stage 5 cells derived according to embodiments of the invention are utilized and differentiated into Stage 6 cells, while in other embodiments Stage 5 cells derived according to other protocols may be utilized.

One aspect of the invention provides methods of enhancing expression of HB9 by treating Stage 4 to Stage 6 cells in a medium comprising T3/T4 or an ALK5 inhibitor or combinations thereof. The Stage 4, Stage 5 and Stage 6 cells may be pancreatic foregut precursor cells, pancreatic endoderm/endocrine precursor cells, and pancreatic endocrine cells, respectively. In some embodiments, the treated population of cells expresses at least two times as much HB9 protein as non-treated cultures. In other embodiments, the level of expression of insulin is positively affected in treated cultures as compared to untreated cultures. However, expression of somatostatin, ghrelin, and glucagon is decreased in treated vs. non-treated cultures. In additional embodiments, Stage 5 cells do not substantially express CDX2 or SOX2.

In further embodiments, the present invention relates to a stepwise method of differentiating pluripotent cells comprising culturing Stage 4 to Stage 6 cells in a media comprising sufficient amounts of T3/T4 or ALK5 inhibitor, or combinations thereof, to generate a population of pancreatic endoderm lineage cells positive for NKX6.1, PDX1, and HB9 protein. In other embodiments, at least 5% of PDX1 and NKX6.1 co-positive cells express HB9 protein. In yet other embodiments, at least 10% of PDX1 and NKX6.1 co-positive cells express HB9 protein. In alternate embodiments, at least 20% of PDX1 and NKX6.1 co-positive cells express HB9 protein. In other embodiments, at least 30% of PDX1 and NKX6.1 co-positive cells express HB9 protein. In alternate embodiments, at least 40% of PDX1 and NKX6.1 co-positive cells express HB9 protein. In other embodiments, at least 50% of PDX1 and NKX6.1 co-positive cells express HB9 protein. In yet other embodiments, at least 60% of PDX1 and NKX6.1 co-positive cells express HB9 protein. In alternate embodiments, at least 70% of PDX1 and NKX6.1 co-positive cells express HB9 protein. In other embodiments, at least 80% of PDX1 and NKX6.1 co-positive cells express HB9 protein. In yet other embodiments, at least 90% of PDX1 and NKX6.1 co-positive cells express HB9 protein. In alternate embodiments, at least 91%, 92%, 93%, 94%, 95%, 96%, 97%, 98%, or 99% of PDX1 and NKX6.1 co-positive cells express HB9 protein.

In some embodiments, a pancreatic endoderm lineage cell population consisting of PDX1, NKX6.1, and HB9 protein positive cells is transplanted into diabetic animals for further in vivo maturation to functional pancreatic endocrine cells. In another embodiment, the invention also encompasses insulin and NKX6.1 expressing cells prepared by the methods of the invention. In yet another embodiment, the invention encompasses a step-wise process of differentiating precursor cells such as pluripotent stem cells into cells of pancreatic endoderm lineage expressing HB9. The methods of invention include one or more of these steps. In particular, the method encompasses the step of differentiating pluripotent stem cells into cells expressing markers characteristic of definitive endoderm cells. This step may take approximately three days. These cells are then differentiated into cells expressing markers characteristic of gut tube cells by culturing the cells under appropriate conditions. In one embodiment, the cells may be cultured for approximately two days. The cells expressing markers characteristic of gut tube cells are then differentiated into cells expressing markers characteristic of foregut endoderm cells. This differentiation may be achieved by culturing the cells for approximately two days. In further embodiments, the pluripotent stem cells are human embryonic pluripotent stem cells.

These cells are then differentiated into cells expressing markers characteristic of pancreatic foregut precursor cells, which in turn may then be differentiated into cells expressing markers characteristic of pancreatic endoderm/endocrine precursor cells, which in turn may then be differentiated into cells expressing markers characteristic of pancreatic endocrine cells. To achieve differentiation into pancreatic endoderm lineage cells expressing HB9, the cells expressing markers characteristic of pancreatic foregut precursor cells, and pancreatic endoderm/endocrine precursor cells may be cultured with one or more of an activin receptor inhibitor (preferably an ALK5 inhibitor), and/or a T3/T4 thyroid hormone. In one embodiment, the cells expressing markers characteristic of pancreatic foregut precursor cells, and pancreatic endoderm/endocrine precursor cells are cultured with T3/T4. In another embodiment, the cells expressing markers characteristic of pancreatic foregut precursor cells, and pancreatic endoderm/endocrine precursor cells are cultured with an activin receptor inhibitor. In an alternate embodiment, the cells are cultured with both an activin receptor inhibitor and T3/T4. The methods of the invention are suitable for any cells that may be differentiable into cells of pancreatic endoderm lineage expressing HB9. Table III illustrates exemplary culture conditions suitable for use in embodiments of methods of the invention. As used in Table III below, “MCX” is MXC compound, “AA” is activin, “ALK5 inh.” is ALK5 inhibitor, “RA” is retinoic acid, “Vit. C” is ascorbic acid, “inh.” is inhibitor, and “act.” is activator. In certain embodiments, any one of the treatments at one stage (e.g. any one of Stage 1, 2, 3, 4, 5 or 6) may be combined with any one of the treatments at another stage (e.g. any one of Stage 1, 2, 3, 4, 5 or 6).

TABLE III
Exemplary culture conditions suitable for use in embodiments of the methods of the invention

| | Stage 1 | Stage 2 | Stage 3 | Stage 4 | Stage 5 | Stage 6 |
|---|---|---|---|---|---|---|
| Treatment of | Pluripotent | Stage 1 cells | Stage 2 cells | Stage 3 cells | Stage 4 cells | Stage 5 cells |
| | stem cells | | | | | |
| With at least | AA & Wnt3A | | | | | |
| | GDF8 & MCX | | | | | |
| | | FGF7 & Vit. C | | | | |
| | | | SANT-1, RA & | | | |
| | | | Noggin | | | |
| | | | FGF7, retinoic acid, | | | |
| | | | SANT-1, a PKC act. | | | |
| | | | (e.g. TPB), a BMP inh. | | | |
| | | | (e.g. LDN-193189), & | | | |
| | | | Vit. C | | | |
| | | | | T3 | ALK5 inh. + T3 | T3 |
| | | | | T3 | ALK5 inh. + T3 | ALK5 inh. + T3 |
| | | | | T3 | T3 | T3 |
| Other | | | | One or more of: | One or more of: | One or more of: |
| optional | | | | SANT-1 | SANT-1 | SANT-1 |
| components | | | | RA | RA | RA |
| | | | | Vit. C | Vit. C | Vit. C |
| | | | | FGF7 | | |
| | | | | BMP Receptor | | |
| | | | | Inh. | | |
| | | | | (e.g. LDN-193189) | | |
| | | | | PKC act. | | |
| | | | | (e.g. TPB) | | |
| Duration of | Approximately | Approximately | Approximately | Approximately | Approximately | Approximately |
| Treatment | 2-5 days; | 2-3 days; | 2-4 days, | 2-4 days, | 2-4 days, | 2-4 days, |
| | preferably | preferably | preferably | preferably | preferably | preferably |
| | about 3-4 days | about 2 days | about 2 days | about 3 days | about 3 days | about 3 days |

In an embodiment, the present invention provides a method of enhancing expression of HB9 by culturing a population of pancreatic endoderm lineage cells in media comprising T3. In some embodiments, the population of pancreatic endoderm lineage cells does not substantially express CDX2 or SOX2. In other embodiments, the population of pancreatic endoderm lineage cells is obtained by a stepwise differentiation of pluripotent cells. In additional embodiments, the pluripotent cells are human embryonic pluripotent cells.

In an embodiment, the present invention provides a method of enhancing expression of HB9 by culturing a population of pancreatic endoderm lineage cells in a medium comprising ALK5 inhibitor. In some embodiments, the population of pancreatic endoderm lineage cells is obtained by a stepwise differentiation of pluripotent cells. In some embodiments, the pluripotent cells are human embryonic pluripotent cells.

In a preferred embodiment, the present invention relates to a method of enhancing expression of HB9 by culturing a population of pancreatic endoderm lineage cells in a medium comprising an ALK5 inhibitor and T3. In some embodiments, the population of pancreatic endoderm lineage cells is obtained by a stepwise differentiation of pluripotent cells. In additional embodiments, the pluripotent cells are human embryonic pluripotent cells.

In another embodiment, the invention refers to a method of enhancing expression of HB9 in PDX1 and NKX6.1 co-expressing cells by treating such cells in a medium comprising a sufficient amount of T3, ALK5 inhibitor or combinations thereof.

One embodiment of the invention is a method for producing cells expressing markers characteristic of β cells from pluripotent stem cells, including the steps of: (a) culturing pluripotent stem cells; (b) differentiating the pluripotent stem cells into cells expressing markers characteristic of foregut endoderm cells; and (c) differentiating the cells expressing markers characteristic of foregut endoderm cells into cells expressing markers characteristic of β cells by treatment with a medium supplemented with T3/T4, an ALK5 inhibitor, or both. The resulting cells may be positive for NKX6.1, PDX1, and Hb-9. The method may be used to enhance the number of HB9 positive cells in NKX6.1 positive cells expressing markers characteristic of pancreatic endoderm precursor cells. The method may also be used to decrease expression of NKX2.2. Moreover, the method suppresses SOX2 and albumin expression. Further, the method may be used to increase the yield of cells expressing insulin.

In one embodiment, T3 is used. The method may include culturing cells in a medium supplemented with T3 and an ALK5 inhibitor. The method may also enhance HB9 expression when compared to cells that are not cultured with a medium supplemented with T3 and an ALK5 inhibitor. The medium may also be further supplemented with any one or more (e.g. 1, 2, 3 or all) of a SMO inhibitor, a SHH signaling pathway antagonist (such as SANT-1), retinoic acid, and ascorbic acid. In one embodiment, the method provides cells expressing markers characteristic of β cells by culturing cells expressing markers characteristic of pancreatic endoderm/endocrine precursor cells in a medium supplemented with T3, which may also be further supplemented with an ALK5 inhibitor.

Another embodiment of the invention is a method of providing cells expressing markers characteristic of β cells including differentiating cells expressing markers characteristic of foregut endoderm cells into cells expressing markers characteristic of β cells by treatment with a medium supplemented with T3/T4, an ALK5 inhibitor, or both. In certain embodiments, the medium is further supplemented with a BMP receptor inhibitor and a PKC activator. The resulting cells are preferably positive for NKX6.1, PDX1, and Hb-9. The method may be used to enhance the number of HB9 positive cells in NKX6.1 positive pancreatic endoderm precursor cells, decrease expression of NKX2.2, and/or suppresses SOX2 and albumin expression. In preferred embodiments, T3 is used. The method may also include culturing cells in a medium supplemented with T3 and an ALK5 inhibitor. The method may also enhance HB9 expression when compared to cells that are not cultured with a medium supplemented with T3 and an ALK5 inhibitor. Moreover, the method may include formation of cells expressing markers characteristic of β cells by culturing cells expressing markers characteristic of pancreatic endoderm/endocrine precursor cells in a medium supplemented with T3 and optionally an ALK5 inhibitor.

Yet another embodiment of the invention is a method of increasing HB9 expression and suppressing SOX2 and albumin expression by culturing cells expressing markers characteristic of pancreatic foregut precursor cells in a medium supplemented with T3/T4 and an ALK5 inhibitor. An alternate embodiment of the invention is a method of down-regulating glucagon, somatostatin and ghrelin in cells expressing markers characteristic of pancreatic foregut precursor cells, cells expressing markers characteristic of pancreatic endoderm/endocrine precursor cells, or cells expressing markers characteristic of endocrine cells, comprising culturing the cells in a medium supplemented with T3/T4 and an ALK5 inhibitor. The medium may be further supplemented with one or more of a SMO inhibitor, a SHH signaling pathway antagonist (such as SANT-1), retinoic acid, and ascorbic acid. In one embodiment, the cells are Stage 4 cells and the medium is further supplemented with FGF7.

The invention also provides a cell or population of cells obtainable by a method of the invention. The invention also provides a cell or population of cells obtained by a method of the invention.

The invention provides methods of treatment. In particular, the invention provides methods for treating a patient suffering from, or at risk of developing, diabetes.

The invention also provides a cell or population of cells obtainable or obtained by a method of the invention for use in a method of treatment. In particular, the invention provides a cell or population of cells obtainable or obtained by a method of the invention for use in a method of treating a patient suffering from, or at risk of developing, diabetes.

The diabetes may be Type 1 or Type 2 diabetes.

In one embodiment, the method of treatment comprises implanting cells obtained or obtainable by a method of the invention into a patient.

In one embodiment, the method of treatment comprises
differentiating pluripotent stem cells in vitro into Stage 1, Stage 2, Stage 3, Stage 4, Stage 5 or Stage 6 cells, for example as described herein,
and implanting the differentiated cells into a patient.

In one embodiment, the method further comprises the step of culturing pluripotent stem cells, for example as described herein, prior to the step of differentiating the pluripotent stem cells.

In one embodiment, the method further comprises the step of differentiating the cells in vivo, after the step of implantation.

In one embodiment, the patient is a mammal, preferably a human.

In one embodiment, the cells may be implanted as dispersed cells or formed into clusters that may be infused into the hepatic portal vein. Alternatively, cells may be provided in biocompatible degradable polymeric supports, porous non-degradable devices or encapsulated to protect from host immune response. Cells may be implanted into an appropriate site in a recipient. The implantation sites include, for example, the liver, natural pancreas, renal subcapsular space, omentum, peritoneum, subserosal space, intestine, stomach, or a subcutaneous pocket.

To enhance further differentiation, survival or activity of the implanted cells in vivo, additional factors, such as growth factors, antioxidants or anti-inflammatory agents, can be administered before, simultaneously with, or after the administration of the cells. These factors can be secreted by endogenous cells and exposed to the administered cells in situ. Implanted cells can be induced to differentiate by any combination of endogenous and exogenously administered growth factors known in the art.

The amount of cells used in implantation depends on a number of various factors including the patient's condition and response to the therapy, and can be determined by one skilled in the art.

In one embodiment, the method of treatment further comprises incorporating the cells into a three-dimensional support prior to implantation. The cells can be maintained in vitro on this support prior to implantation into the patient. Alternatively, the support containing the cells can be directly implanted in the patient without additional in vitro culturing. The support can optionally be incorporated with at least one pharmaceutical agent that facilitates the survival and function of the transplanted cells.

Publications cited throughout this document are hereby incorporated by reference in their entirety. The present invention is further illustrated, but not limited, by the following examples.

### EXAMPLES

### Example 1

### Previously Published Protocols Generating Pancreatic Endoderm Population Derived from Human Pluripotent Cells do not Substantially Express HB9 Protein

This example is directed to identification of the expression pattern of HB9 in cells derived from pluripotent stem cells as described in this Example. Cells of the human embryonic stem cell line H1 (passage 40) were seeded as single cells at 1×10⁵ cells/cm² on MATRIGEL™ (1:30 dilution; BD Biosciences, NJ)-coated dishes in MTESR®1 media (StemCell Technologies, Vancouver, Canada) supplemented with 10 μM of Y27632 (Rock inhibitor, Catalog No. Y0503, Sigma-Aldrich, St. Louis, MO). Forty-eight hours post seeding, the cultures were washed with incomplete PBS (phosphate buffered saline without Mg or Ca). The cultures were then differentiated into pancreatic endoderm/endocrine precursor cells as described previously in Diabetes, 61, 2016, 2012. The differentiation protocol used was as follows:
a. 60-70% confluent adherent cultures of undifferentiated H1 cells plated on 1:30 MATRIGEL™ coated surfaces were exposed to RPMI 1640 medium (Invitrogen) supplemented with 0.2% fetal bovine serum (FBS) (Hyclone, Utah), 100 ng/ml activin-A (AA; Pepro-tech; Rocky Hill, NJ), and 20 ng/ml of Wnt3A (R&D Systems) for day one only. For the next two days, the cells were cultured in RPMI with 0.5% FBS and 100 ng/ml AA.
b. The cells resulting from (a) were exposed to DMEM-F12 medium (Invitrogen) supplemented with 2% FBS and 50 ng/ml of FGF7 (Pepro-tech) for three days.
c. The cultures resulting from (b) were continued for four days in DMEM-HG medium (Invitrogen) supplemented with 0.25 μM SANT-1 (Sigma-Aldrich; St. Louis, MO), 2 μM retinoic acid (Sigma-Aldrich), 100 ng/ml of Noggin (R&D Systems), and 1% (v/v) of a supplement sold under the trademark B27® (Catalogue #17504044, Life Technologies Corporation, Grand Island, NY).
d. Cells resulting from (c) were cultured for three days in DMEM-HG medium supplemented with 1 μM ALK5 inhibitor (ALK5i; Farmingdale, NY), 100 ng/mL of Noggin, 50 nM TPB ((2S,5S)-(E,E)-8-(5-(4-(Trifluoromethyl)phenyl)-2,4-pentadienoylamino)benzolactam; EMD Chemicals Inc., Gibbstown NJ) and 1% B27 in monolayer format. For the last day of culture, cells were treated with 5 mg/mL Dispase for 5 min, followed by gentle pipetting to mix and break into cell clumps (<100 micron). The cell clusters were transferred into disposable polystyrene 125 ml Spinner Flask (Corning), and spun at 80 to 100 rpm overnight in suspension with DMEM-HG supplemented with 1 μM ALK5 inhibitor, 100 ng/ml of Noggin and 1% B27.

At the end of (d), mRNA was collected for PCR analysis of relevant pancreatic endoderm/endocrine genes. Total RNA was extracted with the RNeasy® Mini Kit (Qiagen; Valencia, CA) and reverse-transcribed using a High Capacity cDNA Reverse Transcription Kit (Applied Biosystems, Foster City, CA) according to the manufacturer's instructions. cDNA was amplified using Taqman® Universal Master Mix and Taqman® Gene Expression Assays which were pre-loaded onto custom Taqman® Arrays (Applied Biosystems). The data were analyzed using Sequence Detection Software (Applied Biosystems) and normalized to undifferentiated human embryonic stem (hES) cells using the ΔΔCt method (i.e. qPCR results corrected with internal controls (ΔΔCt=ΔCtₛₐₘₚₗₑ−ΔCt_{reference})). All primers were purchased from Applied Biosystems. FACS and immunofluorescent analysis was done as previously described (Diabetes, 61, 20126, 2012). The HB9 antibody was obtained from Developmental Studies Hybridoma Bank (University of Iowa, Iowa). As used in the Examples, Y27632 ((1R,4r)-4-((R)-1-aminoethyl)-N-(pyridin-4-yl)cyclohexanecarboxamide) is a cell-permeable small molecule Rho-associated kinase (ROCK) inhibitor.

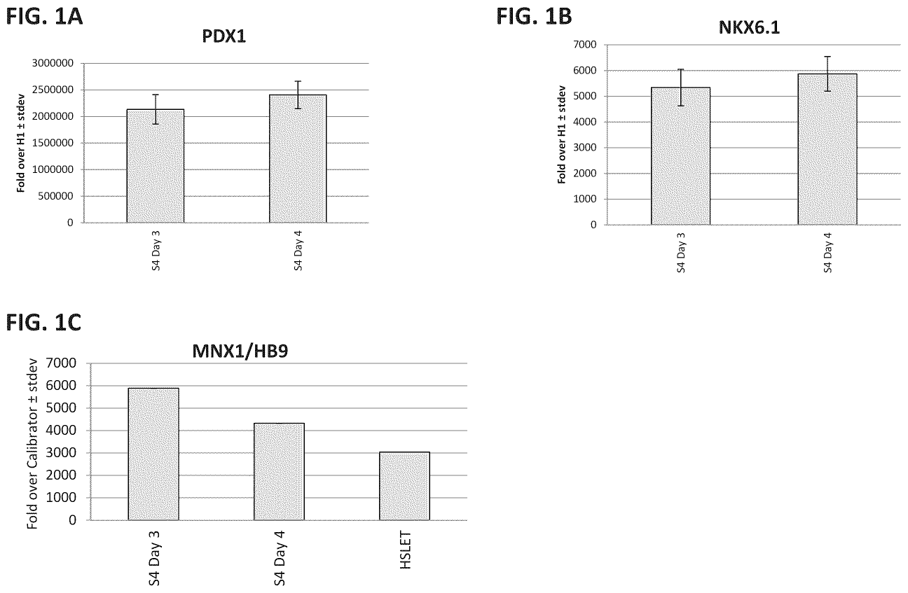

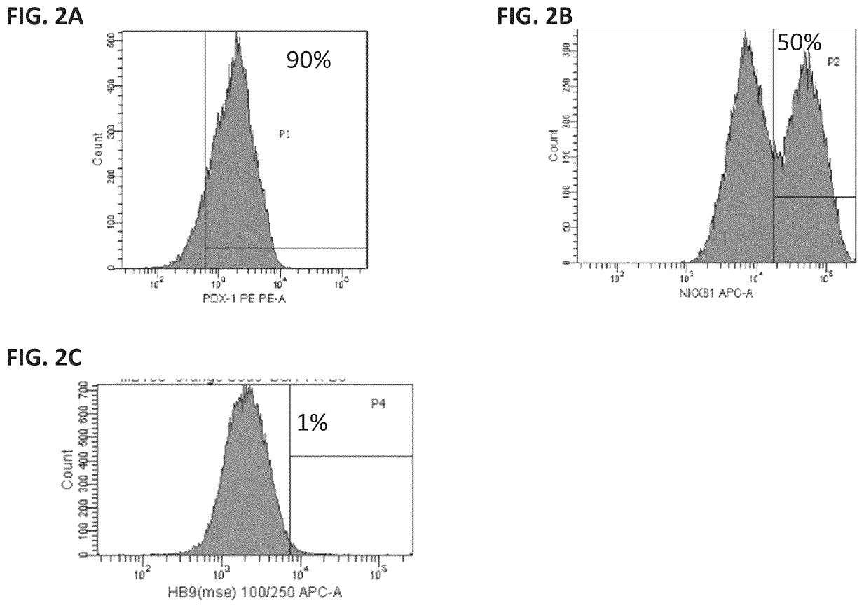

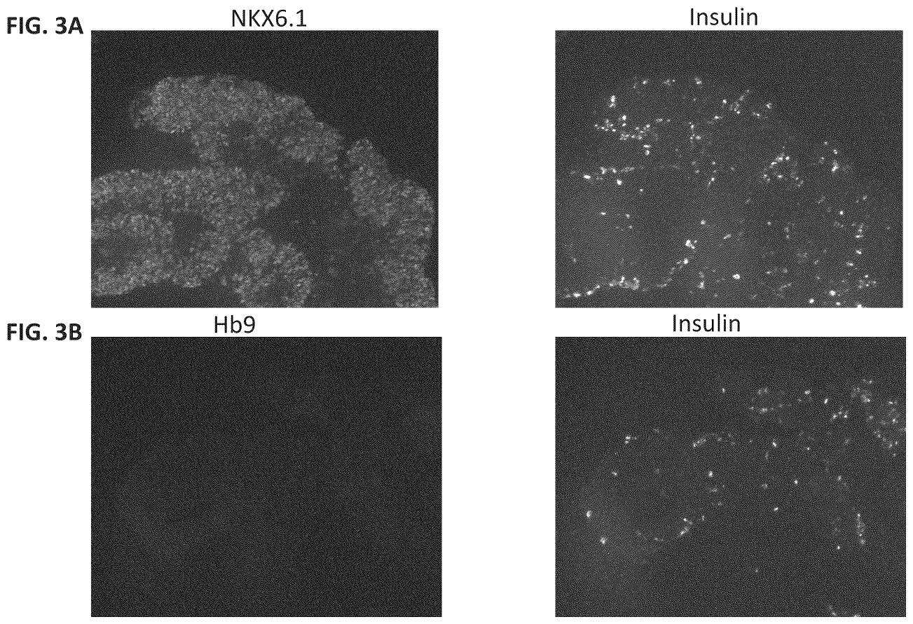

FIGS. 1A to 1C depict data from real-time PCR analyses of the expression of the following genes in cells of the human embryonic stem cell line H1 differentiated to pancreatic endoderm/endocrine precursors as outlined in Example 1: PDX1 (FIG. 1A), NKX6.1 (FIG. 1B), and HB9 (FIG. 1C). As shown in FIGS. 1A-1C, robust mRNA expression of PDX1, NKX6.1, and HB9 was detected in these cultures. Furthermore, mRNA expression of HB9 was equivalent or higher in these cells as compared to human cadaveric islets. However, as shown in FIGS. 2A-2C, whereas gene expression data for PDX1 and NKX6.1 was in accordance with high expression of the corresponding proteins as measured by FACS analysis, mRNA expression of HB9 was discordant with protein expression of HB9. On the day following the completion of (d), i.e., at day 5, approximately 1% of the cells were positive for HB9 whereas approximately 50% of the cells were NKX6.1 positive and approximately 90% were PDX1 positive. Immunostaining of the cell clusters also confirmed the FACS data. As shown in FIGS. 3A-3B, a significant number of NKX6.1 positive cells and few insulin positive cells were present in the clusters. However, there were no HB9 positive cells detected by immunostaining (FIG. 3B).

### Example 2

### Addition of T3 at Stage 4 to Stage 6 Enhances the Number of HB9 Positive Cells

This example is directed to the addition of T3 at Stage 4 to Stage 6 to significantly enhance the number of HB9 positive cells.

Cells of the human embryonic stem cell line H1 (passage 40) were seeded as single cells at 1×10⁵ cells/cm² on MATRIGEL™ (1:30 dilution; BD Biosciences, NJ)-coated dishes in mTeSR®1 media supplemented with 10 μM of Y27632. Forty-eight hours post-seeding, cultures were washed with incomplete PBS (phosphate buffered saline without Mg or Ca). Cultures were differentiated into cells expressing markers characteristic of pancreatic endoderm/endocrine precursor cells by the protocol outlined below.
a. Stage 1 (3 days): The stem cells were cultured for one day in: MCDB-131 medium (Invitrogen Catalog No.10372-019) supplemented with 2% fatty acid-free BSA (Proliant Catalog No. 68700), 0.0012 g/ml sodium bicarbonate (Sigma-Aldrich Catalog No. S3187), 1×GlutaMax™ (Invitrogen Catalog No. 35050-079), 4.5 mM D-glucose (Sigma-Aldrich Catalog No. G8769), 100 ng/ml GDF8 (R&D Systems) and 1 μM of the MCX Compound. The cells were then cultured for an additional day in MCDB-131 medium supplemented with 2% fatty acid-free BSA, 0.0012 g/ml sodium bicarbonate, 1×GlutaMax™, 4.5 mM D-glucose, 100 ng/ml GDF8, and 0.1 μM MCX compound. The cells were then cultured for an additional day in MCDB-131 medium supplemented with 2% fatty acid-free BSA, 0.0012 g/ml sodium bicarbonate, 1×GlutaMax™, 4.5 mM D-glucose, and 100 ng/ml GDF8.
b. Stage 2 (2 days): The Stage 1 cells were then treated for two days with MCDB-131 medium supplemented with 2% fatty acid-free BSA; 0.0012 g/ml sodium bicarbonate; 1×GlutaMax™; 4.5 mM D-glucose; 0.25 mM ascorbic acid (Sigma, MO) and 25 ng/ml FGF7 (R & D Systems, MN).
c. Stage 3 (2 days): The Stage 2 cells were then treated with MCDB-131 medium supplemented with a 1:200 dilution of ITS-X (Gibco® Insulin,-Transferrin-Selenium-Ethanolamine; Invitrogen, Ca); 4.5 mM glucose; 1×GlutaMax™; 0.0017 g/ml sodium bicarbonate; 2% fatty acid-free BSA; 0.25 μM SANT-1 (Sigma, MO); 1 μM RA (Sigma, MO); 25 ng/ml FGF7; 0.25 mM ascorbic acid; 200 nM TPB (PKC activator; Catalog No. 565740; EMD Chemicals, Gibbstown, NJ); and 100 nM LDN (BMP receptor inhibitor; Catalog No. 04-0019; Stemgent) for two days.
d. Stage 4 (3 days): The Stage 3 cells were then treated with MCDB-131 medium supplemented with a 1:200 dilution of ITS-X; 4.5 mM glucose; 1X GlutaMax™; 0.0017 g/ml sodium bicarbonate; 2% fatty acid-free BSA; 0.25 μM SANT-1; 100 nM RA; 2 ng/ml FGF7; 100 nM LDN-193189; 0.25 mM ascorbic acid; and 100 nM TPB for three days.
e. Stage 5 (3 days): The Stage 4 cells were then treated with MCDB-131 medium supplemented with a 1:200 dilution of ITS-X; 4.5 mM glucose; 1X GlutaMax™; 0.0015 g/ml sodium bicarbonate; 2% fatty acid-free BSA; 0.25 μM SANT-1; 50 nM RA; 0.25 mM ascorbic acid; and 500 nM of ALK5 inhibitor SD208 for three days. SD208 is 2-(5-Chloro-2-fluorophenyl)pteridin-4-yl]pyridin-4-yl-amine) having the structure of formula I, and disclosed in Molecular Pharmacology 2007, 72:152-161. SD208 is a 2,4-disubstituted pteridine, ATP-competitive inhibitor of the TGF-βR I kinase.

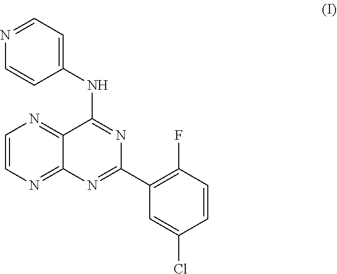 [SMILES: Fc1ccc(Cl)cc1-c1nc(Nc2ccncc2)c2nccnc2n1]

f. Stage 6 (3-15 days): The Stage 5 cells were treated with MCDB-131 medium supplemented with a 1:200 dilution of ITS-X; 4.5 mM glucose; 1X GlutaMax™M; 0.0015 g/ml sodium bicarbonate; 2% fatty acid-free BSA; 0.25 μM SANT-1; 50 nM RA; 0.25 mM ascorbic acid for three days.

In some cultures, 1 μM T3 (T6397, Sigma, MO) was added at Stages 4 through 6. At the end of Stages 4 through 6, the control and treated cultures were analyzed by FACS and immunostaining. Furthermore, mRNA was collected for the control and treated cultures at Stages 2 through 6.

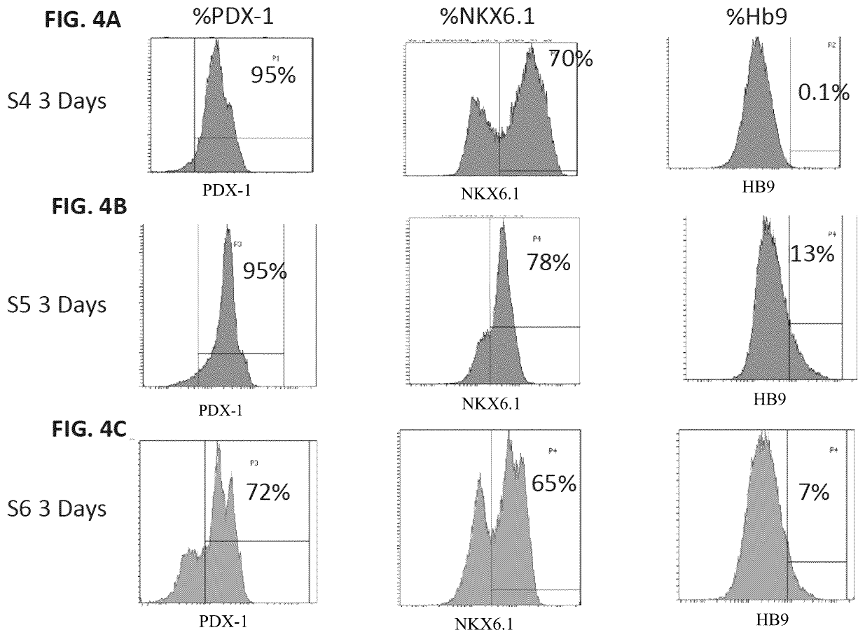

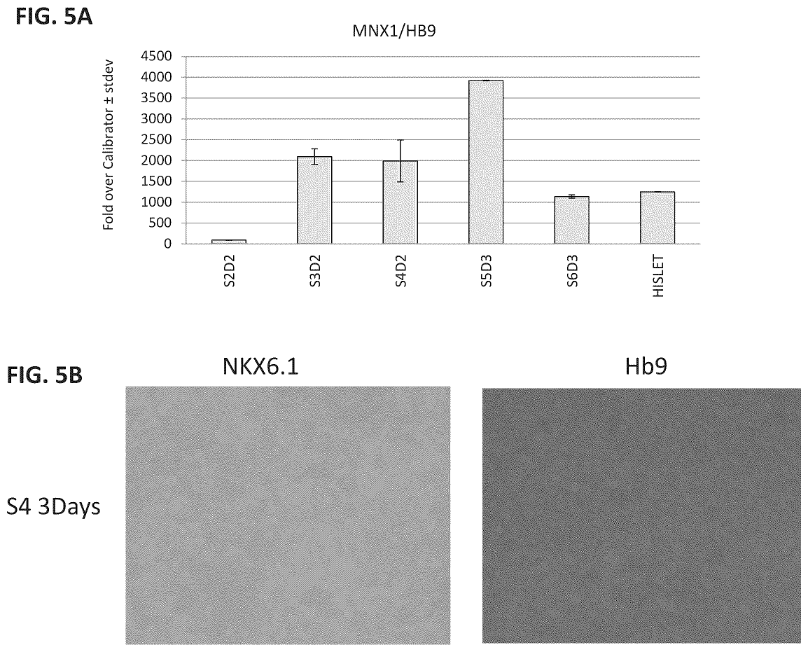

FIGS. 4A-4C depict FACS data at Stage 4 (FIG. 4A), Stage 5 (FIG. 4B), and Stage 6 (FIG. 4C) for PDX1, NKX6.1, and HB9. Consistent with data from Example 1, although there were substantial numbers of PDX1 and NKX6.1 positive cells at Stages 4 through 6, expression of HB9 was far lower. Expression for HB9 peaked at Stage 5 and was diminished at Stage 6. Overall, expression of HB9 for cells generated using the protocol outlined in Example 2 was higher as compared to cells generated using the protocol outlined in Example 1. FIG. 5A shows mRNA expression of HB9 as compared to human islets at Stages 2 through 6. Similar to Example 1, although the mRNA expression level of HB9 at Stages 3 to 4 was equivalent to human islets, HB9 protein expression was very low at Stage 4 (FIG. 5B).

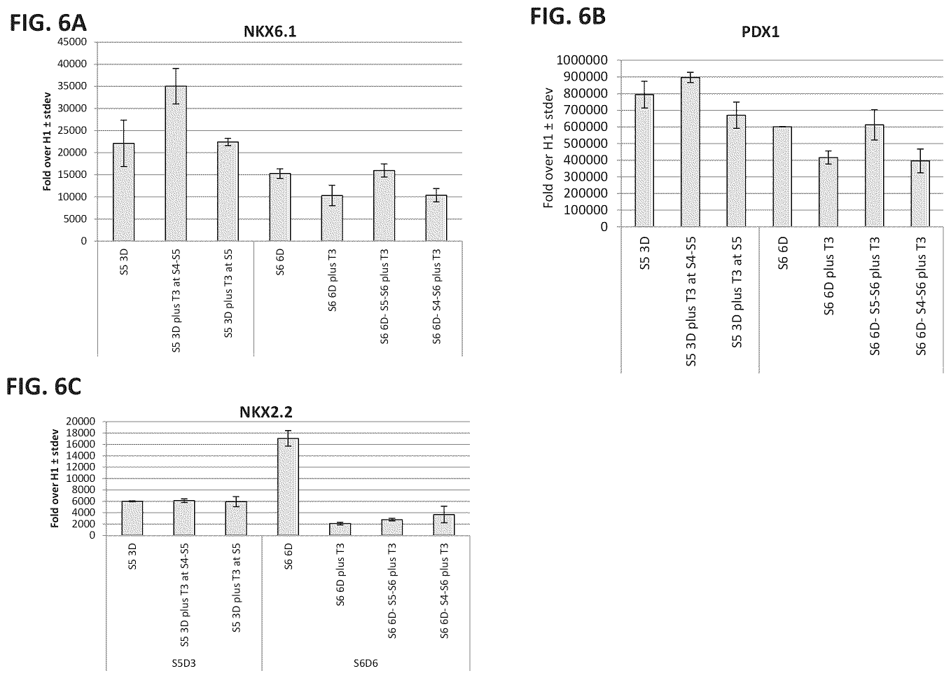

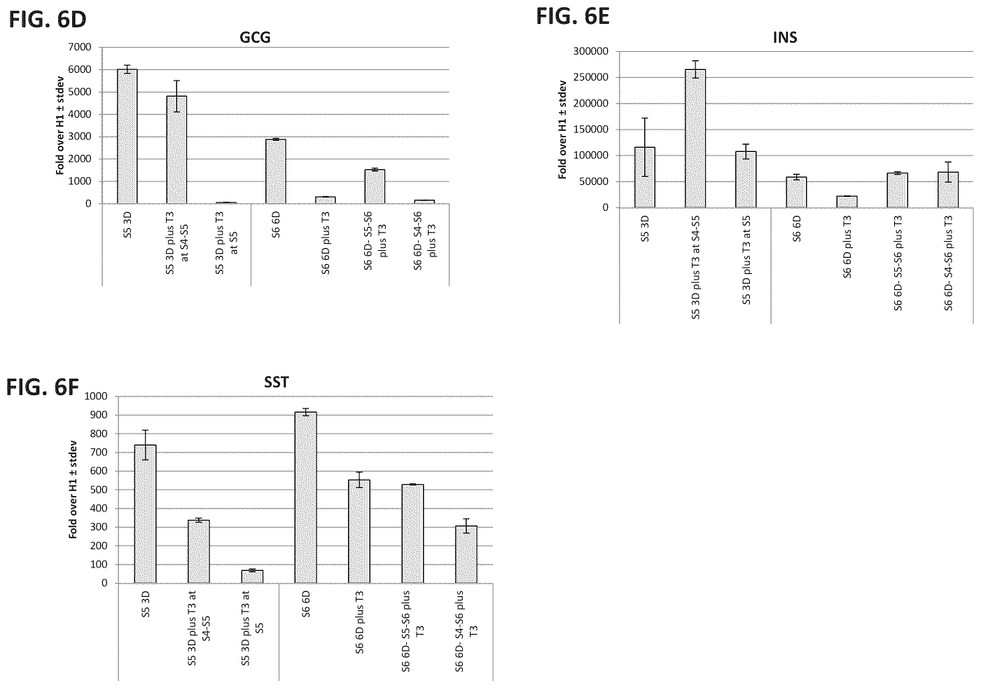

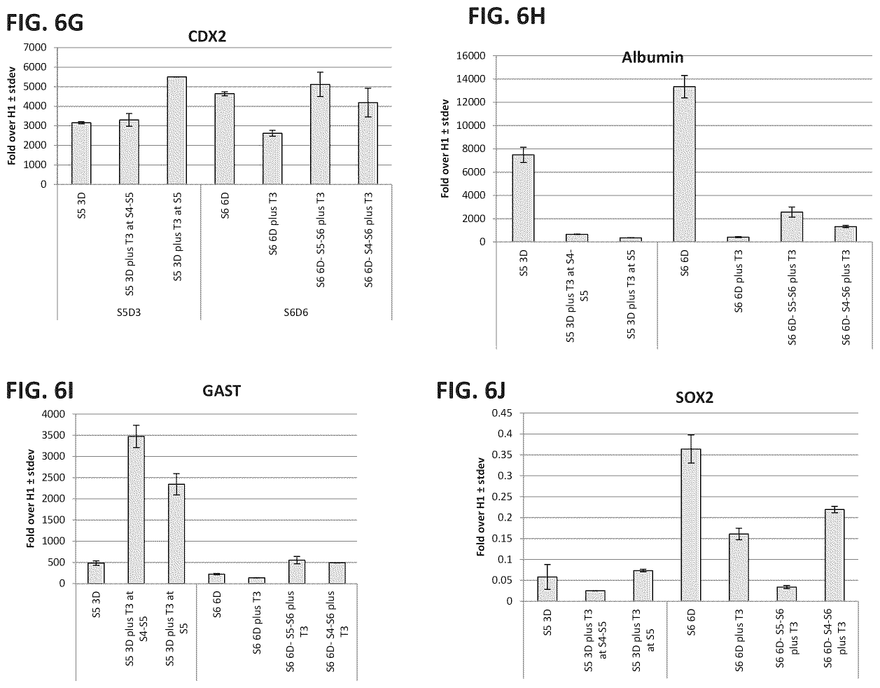

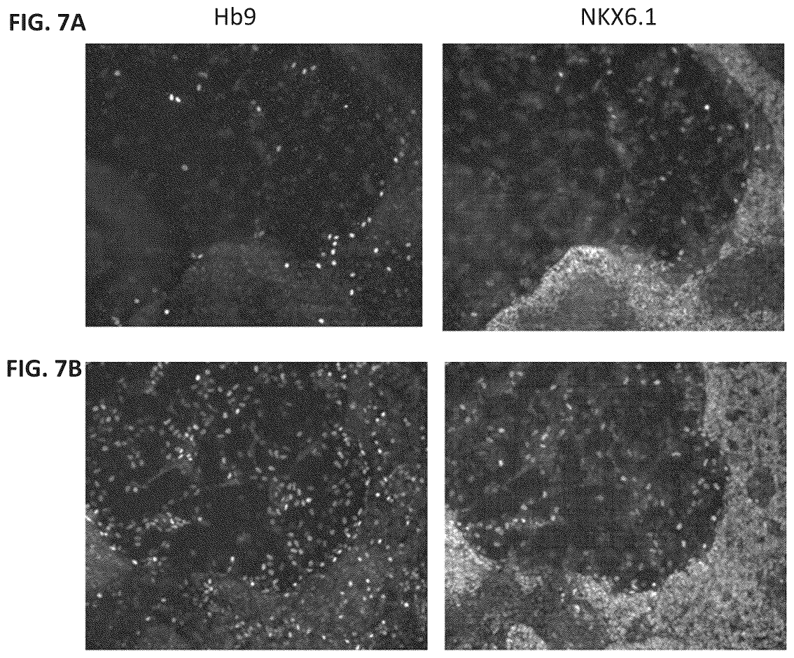

FIGS. 6A to 6J depict data from real-time PCR analyses of the expression of the following genes in cells of the human embryonic stem cell line H1 differentiated to Stage 4 as outlined in Example 2 and treated at Stage 4 only, Stage 4 through Stage 5, or Stage 4 through Stage 6: NKX6.1 (FIG. 6A); PDX1 (FIG. 6B); NKX2.2 (FIG. 6C); glucagon (FIG. 6D); insulin (FIG. 6E); somatostatin (FIG. 6F); CDX2 (FIG. 6G); albumin (FIG. 6H); gastrin (FIG. 6I); and SOX2 (FIG. 6J). The addition of T3 at Stages 4 through 6 significantly down regulated glucagon, somatostatin, and ghrelin while moderately increasing insulin expression at Stage 5. The addition of T3 at Stages 4 through 6 appears to have significantly decreased expression of NKX2.2, while apparently not affecting NKX6.1 and PDX1 expression. Furthermore, T3 addition suppressed SOX2 (stomach marker) and albumin (liver marker) expression while not affecting CDX2 (intestine marker) expression. Immunostaining of control and treated cultures at Stage 6 revealed a significant increase in the number of HB9 positive cells in the T3 treated group (FIG. 7B) as compared to the control (FIG. 7A) at Stage 6. Furthermore, an increased number of NKX6.1 positive cells showed expression of HB9 in T3 treated cultures.

### Example 3

### Combined Treatment with T3 and an ALK5 Inhibitor at Stage 6 Enhances Expression of HB9

This example demonstrates that the combination of an ALK5 inhibitor and T3 in the medium at Stage 6 appears to significantly boost expression of HB9.

Cells of the human embryonic stem cell line H1 (passage 40) were seeded as single cells at 1×10⁵ cells/cm² on MATRIGEL™ (1:30 dilution; BD Biosciences, NJ)-coated dishes in mTeSR®1 media supplemented with 10 μM of Y27632. Forty-eight hours post seeding, cultures were washed with incomplete PBS (phosphate buffered saline without Mg or Ca). Cultures were differentiated into pancreatic endoderm/endocrine lineages by the protocol outlined below.
a. Stage 1 (3 days): The cells were cultured for one day in: MCDB-131 medium (Invitrogen Catalog No.10372-019) supplemented with 2% fatty acid-free BSA (Proliant Catalog No. 68700), 0.0012 g/ml sodium bicarbonate (Sigma-Aldrich Catalog No. S3187), 1×GlutaMax™ (Invitrogen Catalog No. 35050-079), 4.5 mM D-Glucose (Sigma-Aldrich Catalog No. G8769), 100 ng/ml GDF8 (R&D Systems) and 1 μM MCX compound. The cells were then cultured for an additional day in MCDB-131 medium supplemented with 2% fatty acid-free BSA, 0.0012 g/ml sodium bicarbonate, 1×GlutaMax™, 4.5 mM D-glucose, 100 ng/ml GDF8, and 0.1 μM MCX compound. The cells were then cultured for an additional day in MCDB-131 medium supplemented with 2% fatty acid-free BSA, 0.0012 g/ml sodium bicarbonate, 1×GlutaMax™, 4.5 mM D-glucose, and 100 ng/ml GDF8.
b. Stage 2 (2 days): The Stage 1 cells were treated for two days with MCDB-131 medium supplemented with 2% fatty acid-free BSA; 0.0012 g/ml sodium bicarbonate; 1×GlutaMax™; 4.5 mM D-glucose; 0.25 mM ascorbic acid (Sigma, MO) and 25 ng/ml FGF7 (R & D Systems, MN).
c. Stage 3 (2 days): The Stage 2 cells were treated with MCDB-131 medium supplemented with a 1:200 dilution of ITS-X (Invitrogen, Ca); 4.5 mM glucose; 1×GlutaMax™; 0.0017 g/ml sodium bicarbonate; 2% fatty acid-free BSA; 0.25 μM SANT-1 (Sigma, MO); 1 μM RA (Sigma, MO); 25 ng/ml FGF7; 0.25 mM ascorbic acid; 200 nM TPB (PKC activator; Catalog No. 565740; EMD Chemicals, Gibbstown, NJ); and 100 nM LDN (BMP receptor inhibitor; Catalog No. 04-0019; Stemgent) for two days.
d. Stage 4 (3 days): The Stage 3 cells were treated with MCDB-131 medium supplemented with a 1:200 dilution of ITS-X; 4.5 mM glucose; 1×GlutaMax™; 0.0017 g/ml sodium bicarbonate; 2% fatty acid-free BSA; 0.25 μM SANT-1; 100 nM RA; 2 ng/ml FGF7; 100 nM LDN-193189; 0.25 mM ascorbic acid; 100 nM TPB, and 1 μM T3 for three days.
e. Stage 5 (3 days): The Stage 4 cells were treated with MCDB-131 medium supplemented with a 1:200 dilution of ITS-X; 4.5 mM glucose; 1×GlutaMax™; 0.0015 g/ml sodium bicarbonate; 2% fatty acid-free BSA; 0.25 μM SANT-1; 50 nM RA; 0.25 mM ascorbic acid; 1 μM ALK5 inhibitor SD208, and 100 nM T3 for three days.
f. Stage 6 (3-15 days): The Stage 5 cells were treated with MCDB-131 medium supplemented with a 1:200 dilution of ITS-X; 4.5 mM glucose; 1×GlutaMax™; 0.0015 g/ml sodium bicarbonate; 2% fatty acid-free BSA; 0.25 μM SANT-1; 500 nM ALK5 inhibitor, 50 nM RA; 0.25 mM ascorbic acid and 10 nM T3 for three days.

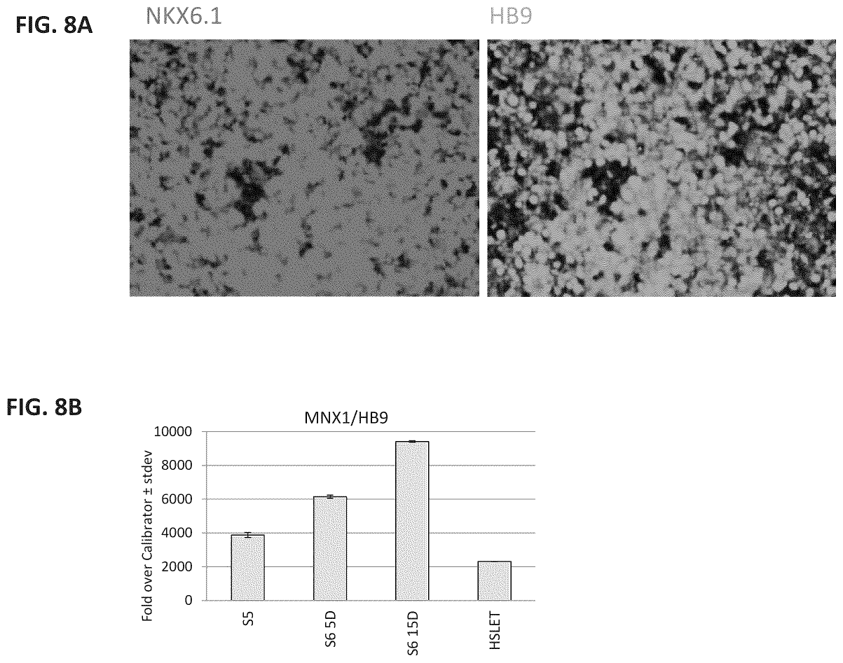

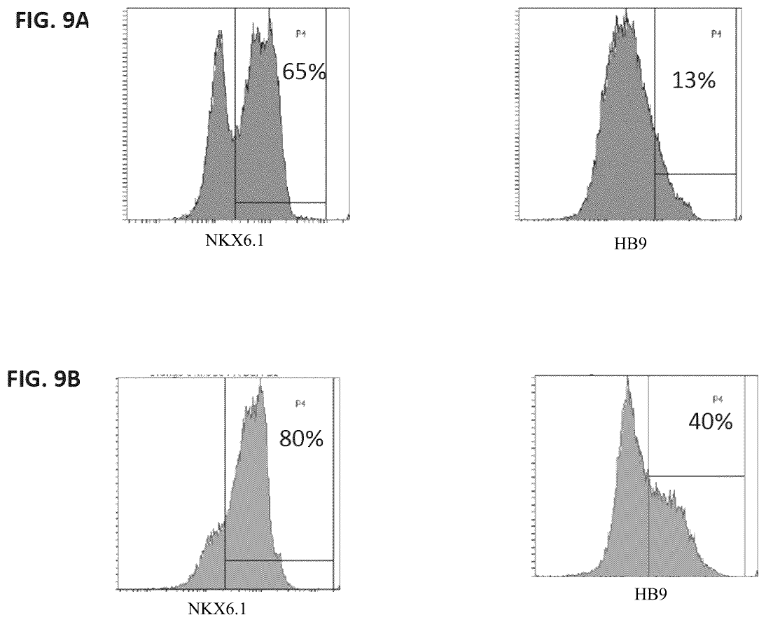

FIGS. 8A and 8B depict immunostaining for NKX6.1 and HB9 at Stage 6 day 7. FIG. 8C depicts data from real-time PCR analyses of the expression of the HB9 in cells of the human embryonic stem cell line H1 differentiated to Stage 6 as outlined in Example 3. The mRNA expression of HB9 along with the immune staining images shows that prolonged exposure to ALK5 inhibitor and T3 appear to significantly enhance expression of HB9 while maintaining robust expression of NKX6.1. FIGS. 9A and 9B depict the FACS data at Stage 6 day 5 and day 15, respectively. A significant fraction of Stage 6 cells show expression of HB9 in Stage 6 day 15 cultures.

### Example 4

### T3 in a Dose Dependent Manner Enhances Expression of HB9

This example shows that T3 in a dose-dependent manner may be used to enhance expression of HB9 while maintaining expression of NKX6.1 at Stage 6. Cells of the human embryonic stem cell line H1 (passage 40) were seeded as single cells at 1×10⁵ cells/cm² on MATRIGEL™ (1:30 dilution; BD Biosciences, NJ)-coated dishes in mTeSR®1 media supplemented with 10 μM of Y27632. Forty-eight hours post-seeding, cultures were washed in incomplete PBS (phosphate buffered saline without Mg or Ca). Cultures were differentiated into cells expressing markers characteristic of pancreatic endoderm/endocrine precursor cells by the protocol outlined below.
a. Stage 1 (3 days): The cells were cultured for one day in: MCDB-131 medium (Invitrogen Catalog No.10372-019) supplemented with 2% fatty acid-free BSA (Proliant Catalog No. 68700), 0.0012 g/ml sodium bicarbonate (Sigma-Aldrich Catalog No. S3187), 1×GlutaMax™ (Invitrogen Catalog No. 35050-079), 4.5 mM D-glucose (Sigma-Aldrich Catalog No. G8769), 100 ng/ml GDF8 (R&D Systems) and 1 μM MCX compound. The cells were then cultured for an additional day in MCDB-131 medium supplemented with 2% fatty acid-free BSA, 0.0012 g/ml sodium bicarbonate, 1×GlutaMax™, 4.5 mM D-glucose, 100 ng/ml GDF8, and 0.1 μM MCX compound. The cells were then cultured for an additional day in MCDB-131 medium supplemented with 2% fatty acid-free BSA, 0.0012 g/ml sodium bicarbonate, 1×GlutaMax™, 4.5 mM D-glucose, and 100 ng/ml GDF8.
b. Stage 2 (2 days): The Stage 1 cells were then treated for two days with MCDB-131 medium supplemented with 2% fatty acid-free BSA; 0.0012 g/ml sodium bicarbonate; 1×GlutaMax™; 4.5 mM D-glucose; 0.25 mM ascorbic acid (Sigma, MO) and 25 ng/ml FGF7 (R & D Systems, MN).
c. Stage 3 (2 days): The Stage 2 cells were then treated with MCDB-131 medium supplemented with a 1:200 dilution of ITS-X (Invitrogen, Ca); 4.5 mM glucose; 1×GlutaMax™; 0.0017 g/ml sodium bicarbonate; 2% fatty acid-free BSA; 0.25 μM SANT-1 (Sigma, MO); 1 μM RA (Sigma, MO); 25 ng/ml FGF7; 0.25 mM ascorbic acid; 200 nM TPB (PKC activator; Catalog No. 565740; EMD Chemicals, Gibbstown, NJ); and 100 nM LDN (BMP receptor inhibitor; Catalog No. 04-0019; Stemgent) for two days.
d. Stage 4 (3 days): The Stage 3 cells were then treated with MCDB-131 medium supplemented with a 1:200 dilution of ITS-X; 4.5 mM glucose; 1×GlutaMax™; 0.0017 g/ml sodium bicarbonate; 2% fatty acid-free BSA; 0.25 μM SANT-1; 100 nM RA; 2 ng/ml FGF7; 100 nM LDN-193189; 0.25 mM ascorbic acid; 100 nM TPB for three days.
e. Stage 5 (3 days): The Stage 4 cells were then treated with MCDB-131 medium supplemented with a 1:200 dilution of ITS-X; 4.5 mM glucose; 1×GlutaMax™; 0.0015 g/ml sodium bicarbonate; 2% fatty acid-free BSA; 0.25 μM SANT-1; 50 nM RA; 0.25 mM ascorbic acid; 1 μM ALK5 inhibitor SD208, and 0-1000 nM T3 for three days.
f. Stage 6 (6 days): The Stage 5 cells were then treated with MCDB-131 medium supplemented with a 1:200 dilution of ITS-X; 4.5 mM glucose; 1×GlutaMax™; 0.0015 g/ml sodium bicarbonate; 2% fatty acid-free BSA; 0.25 μM SANT-1; 500 nM ALK5 inhibitor; 50 nM RA; 0.25 mM ascorbic acid and 0-1000 nM T3 for six days.

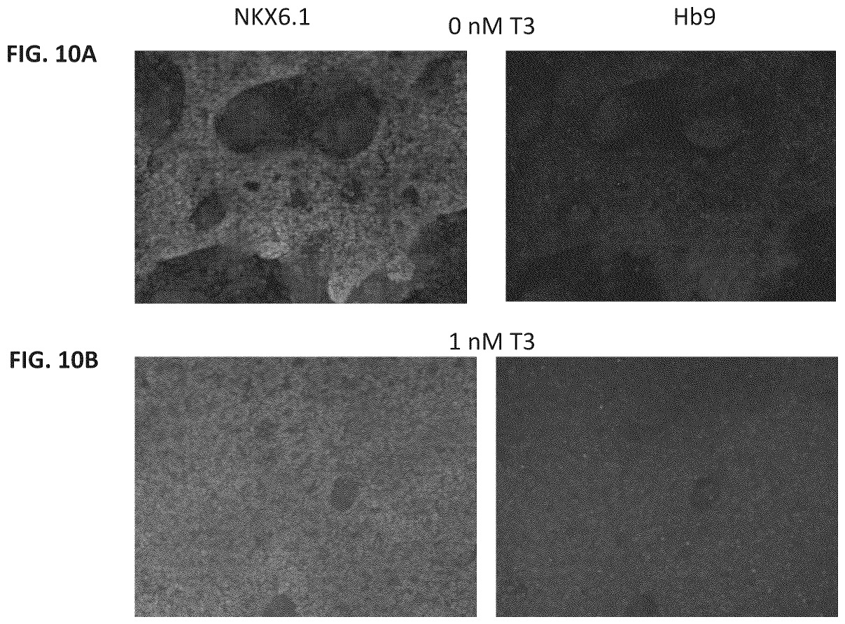

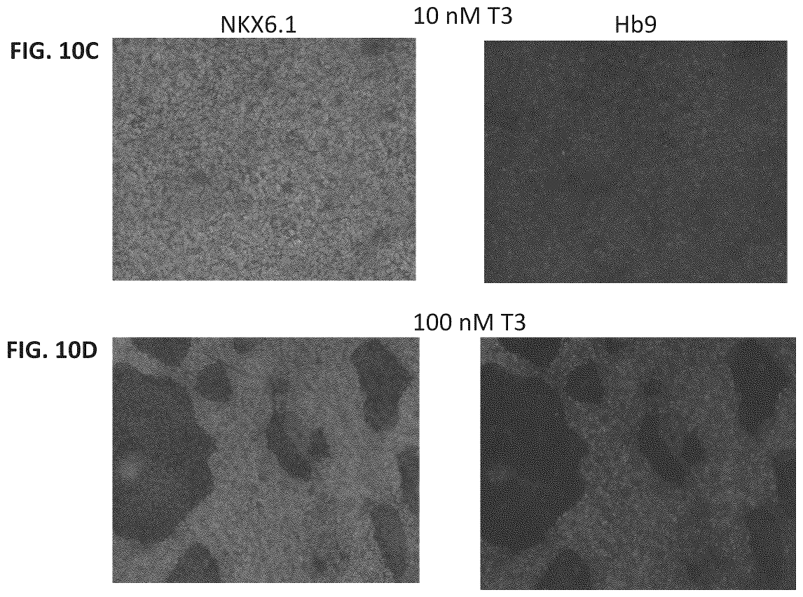

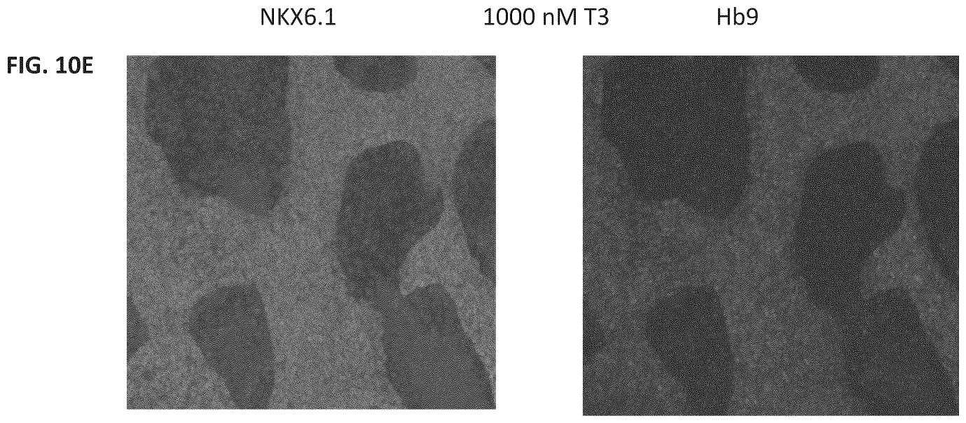

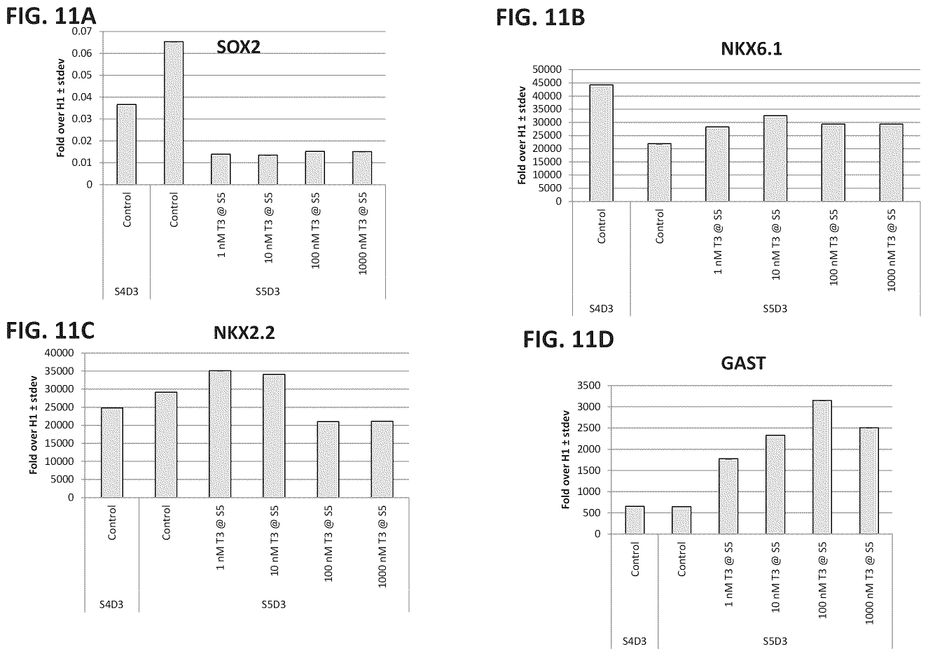

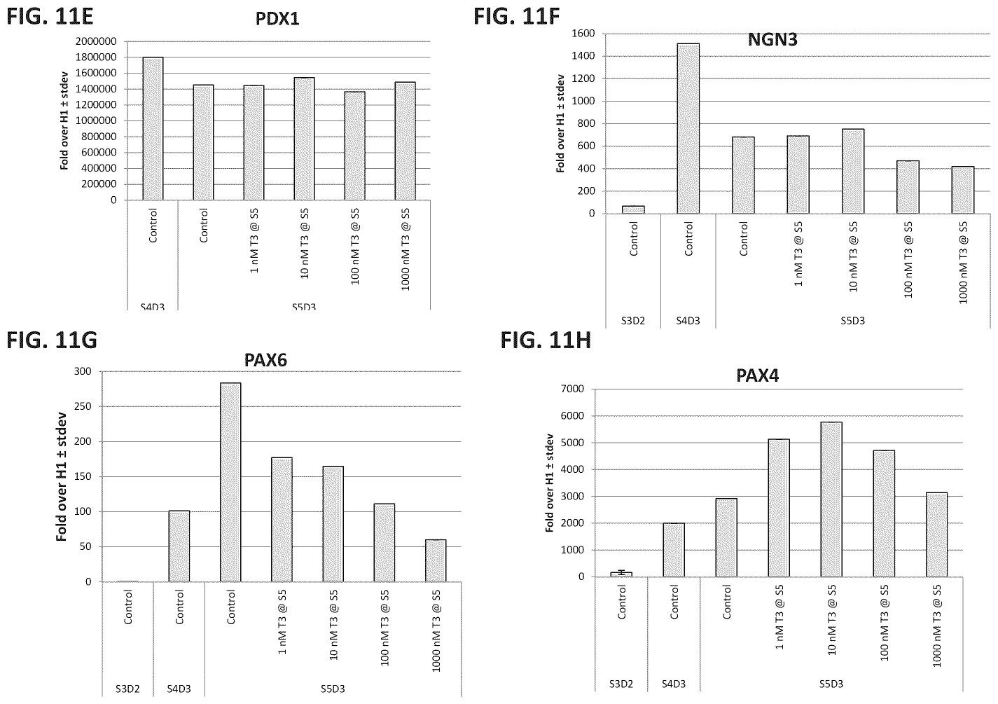

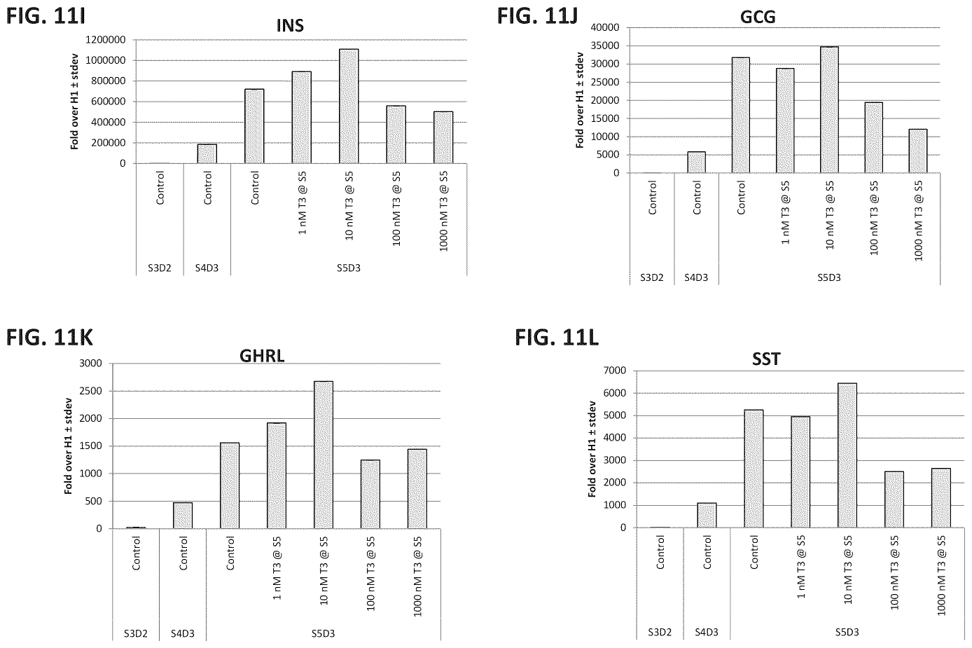

FIGS. 10A to 10E depict immunostaining for NKX6.1 and HB9 at Stage 6 day 6. T3 in a dose dependent manner significantly enhanced the number of HB9 positive cells in the NKX6.1 positive pancreatic endoderm precursor cells. FIGS. 11A to 11L depict data from real-time PCR analyses of the expression of the following genes in cells of the human embryonic stem cell line H1 differentiated to Stage 6 as outlined in Example 4: SOX2 (FIG. 11A); NKX6.1 (FIG. 11B); NKX2.2 (FIG. 11C); gastrin (FIG. 11D); PDX1 (FIG. 11E); NGN3 (FIG. 11F); PAX6 (FIG. 11G); PAX4 (FIG. 11H); insulin (FIG. 11I); glucagon (FIG. 11J); ghrelin (FIG. 11K); and somatostatin (FIG. 11L).

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

1. A method, comprising:
culturing human foregut endoderm cells in a medium supplemented with:
(i) a thyroid hormone, wherein the thyroid hormone is triiodothyronine, thyroxine, an analog of triiodothyronine, an analog of thyroxine or a mixture thereof, or
(ii) both the thyroid hormone and an ALK5 inhibitor.

2. The method of claim 1, wherein the thyroid hormone is triiodothyronine.

3. The method of claim 2, wherein the medium is supplemented with triiodothyronine and the ALK5 inhibitor.

4. The method of claim 1, wherein the medium is further supplemented with SANT-1, retinoic acid, and ascorbic acid.

5. The method of claim 1, wherein the thyroid hormone is thyroxine.

6. The method of claim 5, wherein the medium is supplemented with (ii) the thyroid hormone and the ALK5 inhibitor.

7. The method of claim 6, wherein the ALK5 inhibitor is ALK5 inhibitor II, ALK5i, SD208, TGF-B inhibitor SB431542, ITD-1, LY2109761, A83-01, LY2157299, transforming growth factor (TGF)-β receptor inhibitor V, TGF-β receptor inhibitor I, TGF-β receptor inhibitor IV, TGF-β receptor inhibitor VII, TGF-β receptor inhibitor VIII, TGF-β receptor inhibitor II, TGF-β receptor inhibitor VI, or TGF-β receptor inhibitor III.

8. The method of claim 7, wherein the ALK5 inhibitor is ALK5 inhibitor II.

9. The method of claim 7, wherein the ALK5 inhibitor is A83-01.

10. The method of claim 1, wherein the human foregut endoderm cells are cultured in the medium for a period of from about three to about nine days.

11. The method of claim 10, wherein the human foregut endoderm cells are cultured in a first growth medium supplemented with the thyroid hormone but not the ALK5 inhibitor for a period of from about two to three days, followed by culturing the resulting cell population in an additional growth media supplemented with both the thyroid hormone and the ALK5 inhibitor for about three to six days.

12. The method of claim 11, wherein the human foregut endoderm cells are cultured for a period of about three days in the first growth medium supplemented with the thyroid hormone but not the ALK5 inhibitor.

13. The method of claim 11, wherein the thyroid hormone is triiodothyronine.

14. The method of claim 11, wherein the ALK5 inhibitor is ALK5 inhibitor II, ALK5i, SD208, TGF-B inhibitor SB431542, ITD-1, LY2109761, A83-01, LY2157299, TGF-β receptor inhibitor V, TGF-β receptor inhibitor I, TGF-β receptor inhibitor IV, TGF-β receptor inhibitor VII, TGF-β receptor inhibitor VIII, TGF-β receptor inhibitor II, TGF-β receptor inhibitor VI, or TGF-β receptor inhibitor III.

15. The method of claim 14, wherein the ALK5 inhibitor is ALK5 inhibitor II.

16. The method of claim 14, wherein the ALK5 inhibitor is A83-01.

17. A method, comprising culturing human pancreatic foregut precursor cells in a medium supplemented with triiodothyronine and an ALK5 inhibitor.

18. A method, comprising culturing cells in a medium supplemented with triiodothyronine and an ALK5 inhibitor, wherein the cells are human pancreatic foregut precursor cells, human endocrine precursor cells or human pancreatic endocrine cells.

19. The method of claim 18, wherein the medium is further supplemented with SANT-1, retinoic acid, and ascorbic acid.

20. The method of claim 18, wherein the cells are human pancreatic foregut precursor cells, and wherein the medium is further supplemented with FGF7.
